(19) United States
(12) Patent Application Publication
(10) Pub. No.: US 2014/0295426 A1
(43) Pub. Date: Oct. 2, 2014

(54) Methods for Diagnosing Cancer by Characterization of Tumor Cells Associated with Pleural or Serous Fluids

(71) Applicants: Steven M. Albelda, Philadelphia, PA (US); Daniel Schwed, Philadelphia, PA (US); Chandra Galla Rao, Princeton Junction, NJ (US); Mark Connelly, Doylestown, PA (US); Brad Foulk, Chalfont, PA (US)

(72) Inventors: Steven M. Albelda, Philadelphia, PA (US); Daniel Schwed, Philadelphia, PA (US); Chandra Galla Rao, Princeton Junction, NJ (US); Mark Connelly, Doylestown, PA (US); Brad Foulk, Chalfont, PA (US)

(73) Assignees: VERIDEX LLC, Raritan, NJ (US); THE TRUSTEES OF THE UNIVERSITY OF PENNSYLVANIA, Philadelphia, PA (US)

(21) Appl. No.: 14/235,185

(22) PCT Filed: Jul. 27, 2012
(86) PCT No.: PCT/US2012/048452

§ 371 Date: Apr. 28, 2014

Related U.S. Application Data
(60) Provisional application No. 61/512,576, filed on Jul. 28, 2011.

(51) Int. Cl. G01N 33/574; C12Q 1/68

(52) U.S. Cl. CPC G01N 33/574; C12Q 1/6841
USPC 435/6.11; 435/7.23

(57) ABSTRACT

A method for diagnosing or differentially diagnosing a cancer characterized by the presence of cancer cells in the pleural fluid of a mammalian subject, the method comprising contacting a sample of pleural fluid of the subject with colloidal magnetic particles coupled to a ligand which binds to a determinant on a cancer cell, but does not bind above a baseline threshold to other cellular and non-cellular components in pleural fluid; subjecting the pleural fluid-magnetic particle mixture to a magnetic field to produce a cell fraction enriched in ligand coupled-magnetic particle-bound cancer cells, if present in the pleural fluid; and analyzing the enriched fraction for the number of cancer cells in the pleural fluid. In certain aspects, this method involves preparing the pleural fluids for the above-noted method steps by, e.g., dilution of unprocessed pleural fluid. In certain aspect, the pleural fluid is subjected to the diagnostic method within 24 hours of withdrawal from the subject. This method has advantages to present diagnostic procedures for identifying malignant pleural effusions. The tumor cells present in pleural fluid can be characterized with cellular and molecular markers to determine prognostic and predictive factors.

### BACKGROUND OF THE INVENTION

Normal pleural fluid is a thin film of serous fluid containing small numbers of white blood cells, a variety of proteins, and water, located between the visceral and parietal pleurae (i.e., the membranes lining the lungs and the chest cavity, or the pleural space). A pleural effusion is an abnormal accumulation of fluid in pleural space, which occurs in approximately 1.3 million people each year in the US. There are many causes of pleural effusions (see, Hooper, C et al, 2010 Thorax, Vol. 65 (Suppl 2):ii4-ii17). For example, heart failure or cirrhosis can cause an imbalance between the pressure within blood vessels and the amount of protein in the blood, resulting in an accumulation of fluid, e.g., a transudate. Injury or inflammation of the pleurae may cause abnormal collection of fluid, e.g., an exudate. Causes of exudates include infectious disease, bleeding disorders or trauma, inflammatory lung diseases (e.g., asbestosis), sarcoidosis or autoimmune disorders, cancers, cardiac bypass surgery, heart or lung transplantation, and pancreatitis. However, the three most common causes of pleural effusions are infections, heart failure and malignancy.

In addition to lung cancers, e.g., non-small cell lung cancer (NSCLC) or small cell lung cancer (SCLC), among others, pleural effusions can be a symptom of lymphoma, mesothelioma, and metastatic cancer originating in other organs. Virtually any malignancy can metastasize to the pleural space, but the most common sources of malignant pleural effusions are lung cancer, breast cancer, ovarian cancer, gastrointestinal cancer, lymphomas and mesotheliomas.

Currently, pleural fluid analysis occurs when a patient has a combination of symptoms, although some pleural effusions are asymptomatic. Transudates are normally identified by physical characteristics, e.g., clear fluid, decreased protein or albumin levels, and very low cell counts. Once identified as a transudate, no further testing is done; only follow-up is performed because such pleural effusions are not often due to malignancies. Exudates are normally identified by physical characteristics, e.g., milky, cloudy or reddish appearance due to the presence of lymphatic involvement, the presence of microorganisms or increases in the number of white blood cells or red blood cells. The most important tests to differentiate transudates from exudates are high total protein levels and high LDH levels (Light, R W, 2002 N. Engl. J. Med., Vol. 346(25):1971-1977). Also potentially useful are tests for lactate, amylase, triglyceride and tumor markers.

Typically, diagnosis of a malignant pleural effusion (MPE) is made as summarized below (see, also, Light 2002 and Hooper, 2010, both cited above, incorporated by reference herein). Pleural fluid is obtained and is sent to a cytology laboratory for evaluation. In the laboratory, a representative portion of the specimen is centrifuged and a number of cytologic preparations (including a cell block when possible) are created and stained for diagnostic evaluation. These slides, containing a few thousand cells, are evaluated microscopically. The cytopathologist looks for specific features characteristic of malignancy (i.e., large size, abnormal nuclear features, etc.). If enough cells with these abnormal features are seen, a diagnosis of a malignancy is made. Occasionally, immunocytochemical staining is used.

The sensitivity of a definitive diagnosis based on cytology varies with the cell type and stage of the underlying malignancy and is estimated to about 60% (See, e.g., Hooper 2010, cited above). In a substantial number of cases, a definitive cytological and symptomological diagnosis cannot be made for a pleural effusion. Obstacles to a definitive diagnosis include tumors with relatively “bland” nuclear features, overlapping morphologic features of reactive mesothelial cells, or low numbers of tumor cells present in the pleural fluid at the time of testing. Generally immunocytochemical staining may not be performed due to undetectable numbers of tumor cells or missed cytological identification of tumor cells. Immunocytochemical staining (e.g., an electrochemiluminescent assay) with certain tumor markers has not proven to be diagnostically useful (Hooper, 2010, cited above; and Porcel et al, 2004 Chest, 126:1757-63).

Because there are a number of possible causes of pleural effusion, if the cytology is negative, the patient is generally followed clinically for progression of symptoms. If the cytology is negative with a high clinical suspicion of malignancy, e.g., an undiagnosed effusion with high protein and LDH levels (exudative effusion), the patient may be followed or may be subjected to repeat thoracentesis, followed by repeat cytologic and immunocytochemical analysis at a later time, or a needle pleural biopsy, a thorascopic pleural biopsy, or a surgical pleural biopsy.

Each of these diagnostic options subjects the patients to the danger of undiagnosed, progressing disease and/or the disadvantages of the biopsy procedure. While a pleural biopsy under direct visualization (thorascopy) has a sensitivity and accuracy of diagnosis of 95-100%., it is accompanied by disadvantages including the need for general anesthesia, some morbidity (i.e., infection and pain) and mortality risk, as well as high expense.

### SUMMARY OF THE INVENTION

The methods described herein provide an advantageous alternative to the present methods of diagnosis of conditions related to excess serous fluids such as pleural fluids. These methods can identify malignant cells in pleural effusions and so increase the diagnostic sensitivity and accuracy of the current standard of care. It can also provide access to source material for individualized tumor in vitro growth and/or characterization using clinically relevant cellular and molecular markers

In one aspect, a method is provided for diagnosing or differentially diagnosing a cancer characterized by the presence of cancer cells in the pleural fluid of a mammalian subject. In one aspect, the method includes assaying a pleural fluid sample from a mammalian subject using the CellSearch® technology of Veridex LLC. For example, a sample of the subject's pleural fluid is admixed with colloidal magnetic particles coupled to a ligand (e.g., capture antibody) which binds to a determinant on cancer cells. In one aspect, this first ligand/capture antibody is expressed only on or in cancer cells. In one aspect, this ligand does not bind above a baseline threshold to a determinant expressed on or in other cellular and non-cellular components in pleural fluid other than cancer cells. The resulting pleural fluid-magnetic particle mixture is subjected to a magnetic field to produce a cell fraction enriched in magnetic particle-bound cancer cells, if any are present in the pleural fluid sample. In one embodiment, the enriched fraction is then analyzed for the number of ligand positive cells in the pleural fluid. This ligand positive cell number, if above the baseline threshold, is indicative of the presence of malignant cells in the pleural fluid. A diagnosis or differential diagnosis of a cancer is provided by identifying a number of cancer cells greater than the baseline threshold in the pleural fluid.

In one aspect, the ligand (e.g., capture antibody) employed in this diagnostic method identifies the cell determinant, e.g., EpCAM, and is an anti-EpCAM antibody. In another aspect, the ligand employed in this diagnostic method identifies the cancer cell determinant, e.g., L1CAM, and is an anti-L1CAM antibody. In another aspect, the ligand employed in this diagnostic method identifies the cancer cell determinant, e.g., Claudin 4, and is an anti-Claudin 4 antibody. In one embodiment, the baseline threshold for a diagnosis of malignant pleural effusion is greater than about 100 ligand positive cells/ml of pleural fluid. In other embodiments, the baseline threshold for a diagnosis of malignant pleural effusion is greater than about 500 ligand positive cells/ml of pleural fluid. In other embodiments, the baseline threshold for a diagnosis of malignant pleural effusion is greater than about 1000 ligand positive cells/3.5 ml of pleural fluid. In another aspect, the specificity of this method is enhanced by also staining the EPCAM+ cells for an additional tumor marker, such as the use of “secondary antibodies” to markers such as Cytokeratin, Claudin 4, survivin or telomerase or others, to identify cancer cells present in the pleural fluid. In still other aspects, the specificity of this method using another capture ligand, e.g., L1CAM or Claudin4, can also be enhanced by use of secondary antibodies.

In another aspect, the method employs a cancer cell-type specific ligand and the method is useful to identify the type of cancer cell present in the pleural fluid. In one embodiment, the first ligand (e.g., capture antibody) is an antibody to a cell determinant on mesothelioma cells, e.g., the ligand is an anti-mesothelin antibody, and the cancer identified as mesothelioma. In another aspect, the method employs a cancer cell type specific ligand, e.g., an antibody to a cell determinant on breast cancer cells, e.g., Her2/neu, useful to identifying the type of cancer cell present in the pleural fluid as breast cancer. In another aspect, the method employs a cancer cell type specific ligand, e.g., an antibody to a cell determinant on lung cancer cells, anti-gp160, useful to identifying the type of cancer cell present in the pleural fluid as lung cancer.

In still other aspects, the cancer cell type specific ligand is used as a secondary antibody. In another aspect, these more specific secondary antibodies are used in the method as described herein with the use of the “capture” antibodies to the cancer cell determinant, EPCAM, L1CAM or Claudin4

In another aspect, the method involves enriching a large number of pleural fluid cells to provide a unique source of tumor material, often in quantities that enable the culture, and the morphologic, phenotypic, and molecular characterization of pleural effusion tumor cells (“PETCs”). The cellular and molecular characterization of PETCs enables a broad range of biological, research, and clinical applications for investigation, identification, and individualized classification that can aid clinical management of the personalized treatment of patients and the monitoring of their cancer. Currently, tumor characterization and classification is done on cells of the primary tumor. However, in some MPEs the tissue of origin and location of the primary tumor is not known. In the case of lung cancer in particular, the primary mass is often inaccessible and cannot be biopsied, or the fine needle aspirate provides insufficient material to conduct an adequate tumor evaluation. Finally, in cases of metastatic cancer, the information obtained on the original primary tumor tissue may be months to years old at the time of pleural effusion presentation, and no longer representative of a disease as dynamic as cancer. The ability to enrich large numbers of PETCs can enable tumor characterization at the time of MPE presentation. Some cancer treatments such as hormonal therapy or herceptin in breast cancer or EGFR or ALK inhibitors in lung cancer are based on the genotypic and phenotypic properties of that patient's tumor. PETCs serve as source of tumor material for this important characterization that guides therapeutic choices earlier and more efficiently for patients with MPE.

In another aspect, the method is modified to provide certain preparative steps, e.g., dilution, for the pleural fluid sample before the above method steps are applied.

In another aspect, the method employs a cocktail of markers to identify all non-tumor cells such as CD45, and other antibodies that identify non-tumor cells or combinations thereof.

In another aspect, the method employs prognostic and predictive markers to characterize PETCs including but not limited to EGFR, ER, Ki67, PR, Her2/nu, BCL2, M30, Cox-2, PTEN, IGF-1R, AKT, PARP, CMET, P53, P27, CEA, AR, PSMA, and PSA, etc.

In another aspect, the method enriches PETCs from other non-tumor cells in the exudate for specific molecular characterization of the tumor cells using DNA and RNA markers.

In another aspect, the method enriches PETCs for mutational analysis in genes which include but are not limited to EGFR, BRAF. ARAF, K-ras, and P-53.

In another aspect, the method enumerates PETCs and detects aneusomies, gene amplifications (EGFR), deletions (PTEN, P53) and translocations (i.e., EML4/ALK) by fluorescence in situ hybridization (FISH).

In another aspect, the method isolates live tumor cells for in vitro culture in order to study pharmacokinetics (response to drug treatment), determine the tissue of origin of the tumor, characterize the individual's tumor cells for markers of drug resistance, identify and characterize drug resistant cell populations, phenotypic or molecular molecule expression, mutation analysis, and potential responsiveness to new therapies, and to perform other applications such as discovery of gene signatures.

Other aspects and advantages of the invention are described further in the following detailed description of the preferred embodiments thereof.

### BRIEF DESCRIPTION OF THE FIGURES

FIG. 1A is a graph plotting the number of tumor cells (CK+/CD45−) detected in 3.5 milliliter of pleural fluid of patient samples from the UPCC cohort using the CellSearch® system vs. the diagnosis of pleural effusion etiology, both benign (i.e., congestive heart failure or CHF, end stage renal disease or ESRD, radiation pleuritis, chylothorax) and malignant (lymphoma, mesothelioma, breast cancer, non-small cell lung cancer or NSCLC, squamous cell lung cancer or SCLC, ovarian cancer, and renal cell cancer). Each + represents a different patient pleural fluid sample. A cutoff of about 1100 cells per 3.5 ml of pleural fluid gives 100% specificity.

FIG. 1B is a graph showing the number of CD+/CD45− cells per 3.5 ml of pleural fluid according to histologic type, e.g., benign, malignant, non-epithelial and malignant-epithelial.

FIG. 1C is a graph showing the performance characteristics of the CellSearch® method as discussed in the examples to diagnose malignant pleural effusions, plotting sensitivity vs. specificity, with the area under ROC curve=0.8662. The results are summarized below:

| | | | CORRECT | |
|---|---|---|---|---|
| CUTOFF | SENSITIVITY | SPECIFICITY | CLASSIFICATION | LR |
| 2 | 98.7% | 15.5% | 73.4% | 1.16 |
| 311 | 72.4% | 90.9% | 78% | 7.96 |
| 1509 | 57.8% | 100% | 72.1% | |

FIG. 1D is a graph plotting the number of tumor cells (CK+/Claudin+/CD45−) detected in 3.5 milliliter of pleural fluid of patient samples from the UPCC cohort using the CellSearch® system vs. the diagnosis of pleural effusion etiology, according to histologic type, e.g., benign, malignant, non-epithelial and malignant-epithelial as described in FIG. 1A. Each + represents a different patient pleural fluid sample.

FIG. 1E is a graph showing the number of CD+/Claudin+/CD45− cells per 3.5 ml of pleural fluid according to histologic type, e.g., benign, malignant, non-epithelial and malignant-epithelial.

FIGS. 2A-2G are photographs showing sequential immunostaining of PETC and multicolor FISH images of the same PETC following fixation and hybridization with Satellite Enumeration (SE) probes.

FIG. 2A shows images of PETC stained with Cytokeratin.

FIG. 2B shows images of PETC stained with CD45.

FIG. 2C shows images of PETC stained with DAPI. A large Cytokeratin+/DAPI+ event in the center of the image is the PETC. Arrows indicate location of CD45+ white blood cells.

FIG. 2D shows a FISH image following fixation and hybridization with the SE probe that binds to the centromeres of chromosome 1 (SE-1).

FIG. 2E shows a FISH image following fixation and hybridization with the SE probe that binds to the centromeres of chromosome 7 (SE-7).

FIG. 2F shows a FISH image following fixation and hybridization with the SE probe that binds to the centromeres of chromosome 8 (SE-8). FIG. 2G shows a FISH image following fixation and hybridization with the SE probe that binds to the centromeres of chromosome 17 (SE-17). DAPI counterstain is shown in gray. Copy number gains of all four chromosomes are seen in the PETC. FISH signals on white blood cells (arrows) can be used as hybridization controls.

### DETAILED DESCRIPTION OF THE INVENTION

A method for diagnosing or differentially diagnosing a cancer characterized by the presence of cancer cells in the pleural fluid or serous fluid of a mammalian subject desirably includes assay steps comprising contacting a biological sample of the serous fluid of the subject with colloidal magnetic particles coupled to a ligand which binds to a determinant on cancer cells. The ligand or capture antibody selected for use in this method does not bind above a baseline threshold to other cellular (non-malignant) and non-cellular components in the serous fluid. The serous fluid-magnetic particle mixture is then subjected to a magnetic field to produce a cell fraction enriched in magnetic particle-bound cells in the serous fluid. The enriched fraction is then analyzed to detect the number of ligand-bound (or ligand positive) cells in the serous fluid, as well as other optional information. This ligand positive cell number, if above the baseline threshold, is indicative of the presence of malignant cells in the serous fluid. Depending upon the ligand (e.g., capture antibody) selected, a diagnosis or differential diagnosis of a cancer is provided by identifying a number of ligand-positive cells greater than the baseline threshold for the selected ligand in the serous fluid. As used herein the term “serous fluid” includes, but is not limited to, pleural fluids (such as pleural exudates and pleural transexudates), ascites fluids and the like. An exemplified serous fluid of this invention is pleural fluid.

The diagnostic methods described herein permit a less invasive, more rapid determination of the etiology of a pleural fluid. These diagnostic methods may also significantly lower overall health care costs by reducing the need for repeat thoracentesis and pleural biopsy procedures and providing quicker, more sensitive diagnostic information to a patient for whom cytologic diagnosis was indefinite. Coupled with conventional diagnostic protocols, these methods allow a quicker diagnosis and may permit quicker application of treatment, resulting in a better prognosis for malignant disease. These methods may prevent further worsening of the disease or additional symptoms and complications that may occur during conventional follow up for unknown, indefinite or inaccurate initial diagnosis.

This method employs in its various embodiments, certain method steps and apparatus of Veridex LLC CellSearch® system, which was designed to identify very rare circulating tumor cells (CTC) in blood. For example, in the blood samples normally the subject of the CellSearch® methodology using the ligand to the epithelial cell determinant EpCAM, the baseline threshold of EpCAM positive cells in blood is very low, e.g., about 1 cells/7.5 ml. While this technology has been applied to detection of CTC for a variety of cancers in blood samples (see, e.g., Shaffer D R et al., 2007, Clin Cancer Research, 13:2023), inventors were aware of no suggestions for applying this analytic technique to assist in the diagnosis of any cancer, much less the diagnosis of malignant pleural effusions. There are great differences in the makeup and cellular/non-cellular components of serous fluids, such as pleural fluids and ascites, and blood as well as many differences in the structures of the pleural or abdominal spaces and the vascular system. For example, because the lung is lined with epithelial cells, the “baseline” number of EpCAM-positive cells in pleural fluid is expected to be considerably larger than 1/ml.

### A. THE CELLSEARCH® SYSTEM

The CellSearch Circulating Tumor Cell system (Veridex LLC) employs an assay and apparatus that utilize a combination of immunomagnetic labeling and automated digital microscopy to identify and enumerate the number of rare CTCs in a blood sample. The immunomagnetic labeling is accomplished with the use of a “ferrofluid”, nanoparticles with a magnetic core surrounded by a polymeric layer coated with a ligand specific for an antigenic determinant displayed by the desired cell population, e.g., an antibody for the Epithelial Cell Adhesion Molecule (EpCAM). The ferrofluid-antibody conjugate binds to the antigenic determinant on epithelial cells, which may then be magnetically separated from the remainder of the sample. The cells are then stained with three cellular staining agents to help distinguish epithelial cells from contaminating leukocytes and non-specific debris. The staining agents used are: 4′-6-diamidino-2-phenylindole (DAPI), which is used to stain the nuclei of the cells to help identify viable cells; pycoerythrin (PE)-labeled cytokeratin (CK) antibodies (CK 8, 18, and 19) recognize epithelial cells; and allophycocyanin (APC)-labeled CD45 antibodies bind contaminating leukocytes. Using automated fluorescence-based digital microscopy, the cells are analyzed for the presence of CTCs. CTCs are visualized based on a phenotype of DAM+, CK+, CD45− staining.

Clinical studies using the CellSearch system to analyze peripheral blood have shown that the presence of ≧5 CTCs in patients at baseline or the end of treatment correlated with shorter median progression-free survival and overall survival than patients in all other groups. Patients with <5 CTCs at each time point had the longest median progression-free survival and overall survival. In addition, patients with ≧5 CTCs at baseline, which decreased to <5CTC at the end of treatment had longer median progression-free and overall survival than patients who maintained a CTC count of ≧5. See, e.g., Cristofanilli M, et al 2004 N Engl J Med, 351(8):781-791, which is incorporated herein by reference.

Methods for using the CellSearch system are described in U.S. Pat. Nos. 6,365,362; 6,623,982; 7,282,350; 5,993,665; 6,790,366; and 6,645,731; and in patent applications based thereon. Descriptions of apparatus which may be used to implement the CellSearch system can be found in U.S. Pat. Nos. 5,985,153; 6,861,259; 6,660,159; 6,890,426; 6,136,182 and in patent applications based thereon. Each of the above-noted patents is hereby incorporated by reference in its entirety to describe various aspects of the CellSearch system.

### B. THE SAMPLE AND ITS PREPARATION

The sample for use in the methods described herein is provided by a mammalian subject or patient. Such a subject or patient includes a mammalian animal, e.g., a human, a veterinary or farm animal, a domestic animal or pet, and animals normally used for clinical research, including non-human primates, dogs and mice. Preferably, the subject of these methods is a human. In one aspect of the methods described herein, the subject undergoing the diagnostic method is asymptomatic for a malignancy or a malignant pleural effusion. In another aspect, the subject undergoing the diagnostic methods described herein shows clinical symptoms, or history, of malignancy or a malignant pleural fluid. In still other embodiments, the subject's pleural fluid sample has undergone clinical and cytological study, and optionally cytochemical analysis, resulting in a diagnosis of no detectable malignancy or pleural effusion of unknown etiology.

In one embodiment, the term “biological sample” or “sample” means any pleural fluid or pleural effusion suspected of containing abnormal, malignant cells. Such cells may be derived from a primary or metastatic lung cancer, such as NSCLC or SCLC. Alternatively such cells may be secondary metastatic cancer cells which originated from another organ, e.g., breast, ovary, colon or prostate. In one aspect, the most suitable sample for use in the methods described herein is a pleural exudate. In another aspect, the sample for use in the methods described herein is a pleural transudate. Other biological samples may include other serous fluids containing malignant cells, including, e.g., ascites fluid from the abdomen or pancreatic cyst fluid. Ascites fluid and pleural fluids involve very similar chemical systems; both the abdomen and lung have mesothelial lines and fluid forms in the pleural space and abdominal spaces in the same matter in malignancies. Where in the following disclosure pleural fluid is exemplified, the same methods may be performed with similar results using ascites or other cyst fluids.

In one embodiment, the pleural fluid is used in the method in unprocessed form, directly as removed from the patient. In one embodiment, the unprocessed pleural fluid is placed in a standard blood collection tube, such as an EDTA or Heparin tube, prior to the contacting step. In one embodiment, the unprocessed pleural fluid is placed in a standard CellSave® tube (Veridex) prior to the contacting step. In yet another embodiment, the sample is placed in the CellSave tube immediately after collection from the patient to avoid a decrease in the cancer cells, which may occur to a significant extent within 24 hours, if left in the untreated pleural fluid, even at 4° C. In another embodiment, the sample is placed in the appropriate collection tube within 1 hour, 5 hours, 10 hours, 15 hours, or up to 24 hours after removal from the patient.

In other embodiments of the methods, the sample from the chosen subject may be treated prior to the contact with the CellSearch particles. In one embodiment, the dilution is 1:10 pleural fluid to diluent. In another embodiment, the dilution is 1:9 pleural fluid to diluent. In another embodiment, the dilution is 1:8 pleural fluid to diluent. In another embodiment, the dilution is 1:5 pleural fluid to diluent. In another embodiment, the dilution is 1:2 pleural fluid to diluent. In another embodiment, the dilution is 1:1 pleural fluid to diluent. Preferred diluents include saline, phosphate buffered saline, another buffer or a physiologically acceptable diluent. In one embodiment, the diluted pleural fluid is placed in the CellSave tube before being contacted with the magnetic particle as described in the method herein. In yet another embodiment, the sample is placed in the CellSave tube immediately after collection from the patient and dilution to avoid a decrease in the cancer cells, which may occur to a significant extent within 24-48 hours, if left in the untreated pleural fluid, even at 4° C. In another embodiment, the sample is placed in the appropriate collection tube within 1 hour, 5 hours, 10 hours, 15 hours, 24 hours, 36 hours, up to 48 hours after removal from the patient, and dilution.

In still another embodiment, such samples are concentrated by conventional means prior to the contacting step of the method. This pre-treatment of the pleural fluid is preferable in circumstances in which the pleural fluid must be cryopreserved for shipment to a laboratory performing the method or for later analysis (i.e., later than 24-48 hours post-collection). In such an embodiment, the pleural fluid sample used in the contacting step is prepared by centrifuging the pleural fluid sample after its withdrawal from the subject and resuspending the centrifugate or pellet in buffer. In still other embodiments, the pleural fluid sample is subjected to multiple centrifugations and resuspensions, such as illustrated in Example 1 below, before it is cryopreserved for transport or later analysis.

In another embodiment, such samples are concentrated prior to the contacting step of the method by using a filtration method. In such an embodiment, the pleural fluid sample used in the contacting step is prepared by filtering the fluid through a filter containing a known and essentially uniform pore size that allows for passage of the pleural fluid through the membrane but retains the tumor cells. In such an embodiment, the diameter of the pores in the membrane may be at least 4 μM. In another embodiment the pore diameter may be 5 μM or more, and in other embodiment, any of 6, 7, 8, 9, or 10 μM. After filtration, the tumor cells retained by the membrane may be rinsed off the membrane into a suitable physiologically acceptable buffer. Cells concentrated in this way may then be used in the contacting step of the method.

In another embodiment, the sample, e.g., the untreated pleural fluid, diluted pleural fluid, or the resuspended cell pellet, is contacted with a lytic reagent that differentially lyses non-nucleated red blood cells present in the sample. This step is desirably performed prior to contacting the sample with the magnetic particle in circumstances in which the pleural fluid contains substantial numbers of RBCs. Suitable lysing reagents include a single lytic reagent or a lytic reagent and a quench reagent, or a lytic agent, a quench reagent and a fixation reagent. Suitable lytic systems are marketed commercially and include the BD Pharm Lyse™ system utilized in Example 1 below (Becton Dickenson). Other lytic systems include the Versalyse™ system, the FACSlyse™ system (Becton Dickenson), the Immunoprep™ system or Erythrolyse II system (Beckman Coulter, Inc.), or an ammonium chloride system. The lytic reagent can vary with the primary requirements being efficient lysis of the red blood cells, and the conservation of the antigenic determinants on the cancer cells and on the WBCs in the pleural fluid. In addition to employing a single reagent for lysis, the lytic systems useful in methods described herein can include a second reagent, e.g., one that quenches or retards the effect of the lytic reagent during the remaining steps of the method, e.g., Stabilyse™ reagent (Beckman Coulter, Inc.). A conventional fixation reagent may also be employed depending upon the choice of lytic reagents or the preferred implementation of the method.

In still another embodiment, the pleural fluid sample, unprocessed, diluted or multiply centrifuged or processed as described herein, is cryopreserved at a temperature of about −140° C. prior to being contacted with the colloidal particles described above.

### C. EMBODIMENTS OF THE DIAGNOSTIC METHOD

A variety of embodiments of the general methods described above may be employed in the diagnosis of malignant pleural effusions.

The general diagnostic method, as stated above, employs the steps of collecting the pleural fluid, contacting a sample of pleural fluid of the subject with colloidal magnetic particles coupled to a first ligand which binds to a determinant on cancer cells, but does not bind above a baseline threshold to other cellular and non-cellular components in pleural fluid; subjecting the pleural fluid-magnetic particle mixture to a magnetic field to produce a cell fraction enriched in magnetic particle-bound cancer cells, if present in the pleural fluid; analyzing the enriched fraction for the number of cancer cells in the pleural fluid; and providing a differential diagnosis of a cancer by identifying a number of ligand positive cells greater than the baseline threshold in the pleural fluid. In the alternative, the diagnosis of no malignancy is provided by identifying a number of ligand positive cells lesser than the baseline threshold in the pleural fluid.

In one embodiment of the methods described herein, the diagnostic method includes performing a clinical evaluation of the mammalian subject for clinical indicators of malignancy or a malignant pleural effusion. Such symptoms or indicators include, but are not limited to, chest pain, coughing, difficulty breathing, fatigue, and inflammation. In one embodiment, clinical evaluation is performed before the general diagnostic steps of the method.

In another embodiment, the method of diagnosis includes a step of performing cytologic or immunocytologic or cytochemical examination of the pleural fluid for the presence of abnormal cells before analyzing the pleural fluid by the contacting, subjecting and analysis steps of the method. In this embodiment, the analysis of the pleural fluid provides a diagnostic number of ligand-positive cells, which if it exceeds the baseline, can confirm a cytological diagnosis of malignancy. Alternatively, the analysis may contradict a cytological diagnosis of no malignancy, or an indefinite diagnosis and focuses additional cytological examination of the cells of the pleural fluid. Thus, the diagnostic method may include additional cytological steps or additional surgical diagnostic steps, e.g., biopsy. Alternatively, if the number of ligand-positive cells is well below the baseline, the cytological diagnosis may be confirmed and the method of diagnosis may require no additional steps, or may include further steps to detect infection or one of the non-malignant causes of pleural effusion.

In another embodiment, the method of diagnosis involves performing cytologic or immunocytologic or cytochemical examination of the pleural fluid for the presence of abnormal cells after analyzing the pleural fluid by the contacting, subjecting and analysis steps of the method. In this embodiment, the analysis of the pleural fluid provides a diagnostic number of ligand-positive cells, which if it exceeds the baseline, indicates the need for further cytological examination of the cells of the pleural fluid with cancer-specific reagents to identify or confirm the source of the cancer. Thus, the diagnostic method may include additional cytological steps or indicated that additional surgical steps in diagnosis be employed, e.g., biopsy. Alternatively, if the number of ligand-positive cells is well below the baseline, the method of diagnosis may indicate only additional diagnostic steps to detect infection or one of the non-malignant causes of pleural effusion.

In still another embodiment, as discussed above, the contacting step with the colloidal particle occurs within 24 hours of withdrawing the pleural fluid sample from the subject, if the fluid is not cryopreserved. In still another embodiment, as discussed above, the contacting step with the colloidal particle occurs within 30 hours of withdrawing the pleural fluid sample from the subject. In still another embodiment, as discussed above, the contacting step with the colloidal particle occurs within 48 hours of withdrawing the pleural fluid sample from the subject. In the latter two instances, in certain embodiments the sample is cryopreserved and reconstituted by conventional methods, prior to the contacting step.

In one embodiment, the step of contacting a sample of pleural fluid of the subject with colloidal magnetic particles coupled to a first ligand (e.g., capture antibody) which binds to a determinant on cancer cells employs a ligand that (a) does not bind at all to other cellular and non-cellular components in pleural fluid or (b) does not bind above a baseline threshold to other cellular and non-cellular components in pleural fluid; or (c) binds above a baseline threshold only to certain cancer cell types; or (d) binds to different cancer cell types at a distinguishable number. For example, the ligand employed may be a ligand that binds to a determinant expressed in cancer cells, and not on non-cancer cells. In another embodiment, the ligand employed binds to only cancer cells of a specific cell type, e.g., epithelial cell cancers. In another embodiment, the ligand employed binds to only cancer cells derived from a certain organ, e.g., breast cancer cells, but not from other normal cells or cancer cells from a different origin, e.g., lung cancer cells. In still another embodiment, the ligand binds at distinctly different concentration/numbers to one cancer cell type, e.g., binds in much greater numbers to a breast cancer cell than it does to a lung cancer cell, thereby permitting the cancer type to be identified by the number produced in the analysis step.

In one embodiment the first ligand is a monoclonal antibody or fragment thereof that is capable of binding to a determinant on the cancer cell. By “fragment” is meant a Fab fragment, a Fab′ fragment, or a F(ab′)² fragment of a selected antibody. Similarly a single chain variable antibody fragment or a recombinant construct comprising a complementarity determining region (CDR) of a suitable antibody may be employed as the first ligand useful in these methods. Still other ligands are described in the patent publications discussed herein.

In one embodiment the first ligand is a monoclonal antibody or fragment thereof specific for at least one cancer cell determinant. In one embodiment, as described in the publications and in the Example 3 below, the first ligand is a monoclonal antibody or useful fragment thereof that binds specifically to an epithelial cell adhesion molecule (EpCAM). The EpCAM receptor is not present on WBCs present in the pleural effusions, but it is widely expressed on a variety of carcinomas. However, the EpCAM ligand is not expressed on non-epithelial cancers or on cancer cells of hematologic origin. For example, EpCAM is not present on mesothelioma cancer cells, on leukemias, on lymphomas or on multiple myelomas. When anti-EpCAM is the ligand employed in the method, it may not readily identify possible EpCAM negative renal cell or thymic cell cancers. However, as identified by the examples below, the anti-EpCAM antibody does bind significantly to NSCLC, ovarian cancer and breast cancer. See, e.g., the data of Table 3.

In another embodiment of this method, the first ligand and/or capture antibody binds to another cell specific determinant that is upregulated on highly malignant cells, such as L1CAM. See, e.g., Katayama et al, 1997, Cell Structure and Function 22:511-516; Hai et al, 2012 Clin Cancer Res, 18:1914-1924; and Tischler et al, 2011, Molecul. Cancer, 10:127. In another embodiment, the first ligand or capture antibody binds to the cancer cell determinant Claudin 4.

To improve the specificity of these methods, additional other ligands (e.g., secondary ligands and/or secondary antibodies) are used to confirm malignancy or non-malignancy by staining. In one embodiment, such secondary ligands/antibodies bind determinants or antigens expressed in non-cancer or benign cells, e.g., on WBCs. In another embodiment, if the determinants to which such secondary antibodies bind are tumor specific determinants, the binding of the secondary antibodies can confirm the cells were malignant. If such determinants to which the secondary antibodies bound are expressed only or primarily on benign cells, the secondary antibodies are used in the methods described herein to exclude cells as non-malignant. In one embodiment, such secondary antibodies include antibodies to survivan, telomerase or Claudin 4 (if EpCAM or L1CAM is the capture antibody). In another embodiments of this method and depending upon the cancer cell type in the pleural effusion, the first ligand or secondary ligand is one that binds specifically to a lung cancer cell or to a type of lung cancer cell.

In another embodiment, the first ligand or secondary ligand binds to a cancer cell determinant that is uniquely, or at least differentially, expressed among different lung cancers. Such first antibody/capture antibody or secondary antibodies may be selected from among many known antibodies. Depending on whether the ligands bind to determinants that are primarily expressed on cancer cells, these ligands may be used as the capture antibody or secondary antibodies. One of skill in the art can readily determine by resort to published information, which antibodies can be used as capture or secondary antibodies. For example, in one embodiment, an antibody useful as a ligand for diagnosing non-small cell lung cancer (NSCLC) includes an antibody that binds an EGFR mutation, including anti-delE746-A750 and anti-L858R. In another embodiment, a ligand for use in these methods may be an IgG2Ak antibody such as 703D4 and 704A1. In still another embodiment, the first ligand is an antibody useful for diagnosing small cell lung cancer (SCLC) including, for example the SENT antibody to the NCAM antigen (see, e.g., WO1994/006929). In still other embodiments, the first ligand is an antibody to TTF1, Carcinoembryonic antigen, mMET, MUC1, or Epidermal growth factor receptor. Still other possible antibodies for use as first (capture) ligands or secondary ligands are identified in Table 1 and are known to those of skill in the art. See, e.g., the antibodies and antigens listed in Dennis, J L et al, 2005 Clin. Cancer Res. 11:3766-3772.

In another embodiment the first ligand or secondary ligand is an antibody for diagnosing mesothelioma. In one embodiment of the method, therefore, the first ligand or secondary ligand is an antibody that binds to calretinin. In another embodiment of the method, therefore, the first ligand or secondary ligand is an antibody to tumor 1 (WT1), or toBG8, or to CD15 or to mesothelin.

In still another embodiment, in which the cancer cell present in the pleural effusion is a metastatic cancer cell of the breast, ovary, prostate or colon or other organ, the first ligand or secondary ligands include antibodies of different specificity for those cells. For example, the first ligand or secondary ligand may be monoclonal antibody F36/22 or a fragment thereof that is specific to human breast carcinoma cells (see, e.g., U.S. Pat. No. 5,652,114). In another embodiment, the first ligand or secondary ligand may be a monoclonal antibody directed to an epitope on colorectal cancer cells (see, e.g., U.S. Pat. No. 5,459,043). In another embodiment, the first ligand or secondary ligand may be a monoclonal antibody or fragment thereof that binds an estrogen receptor. In another embodiment, the first ligand or secondary ligand may be a monoclonal antibody or fragment thereof that binds Her2/nu. In another embodiment, the first ligand or secondary ligand may be a monoclonal antibody or fragment thereof that binds prostate specific antigen. Still other embodiments can employ antibodies or fragments thereof to the cell determinants or antigens: CA15.3, CEA, CA125, cancer testes antigens, CDX2, CK20, GCDFP-15, ER, lysozyme, and CK7. Still other antibodies and cell determinants that are useful in the invention are known to those of skill in the art and may be readily selected to identify a particular cancer. See, e.g., the antibodies and antigens that are listed in Table 1 and in e.g., Dennis, J L et al, 2005 Clin. Cancer Res. 11:3766-3772.

In still another embodiment, the method may include more than one first (capture) and/or secondary ligand in the contacting step, wherein each first or secondary ligand is a different antibody (e.g., “labeling reagents” described in the CellSearch technology) directed to a different determinant on the same cancer cell or cancer cell type. In another embodiment, the contacting, subjecting and analyzing steps of the method are repeated, each using a different first/capture or secondary ligand directed to a different determinant on the same cancer cell type. Another embodiment of the method employs first or secondary ligands or a series or panel of first or secondary ligands, which are differentially expressed on different cancer cell types. In another embodiment, the contacting, subjecting and analyzing steps of the method are repeated, each using a different “first ligand” and/or secondary ligand directed to a different determinant on a different cancer cell type. See, e.g., the publications referenced herein. Therefore a combination of different antibodies that bind different determinants on the same cancer cell type or population can be employed in place of a single antibody that binds one determinant on a cancer cell population.

Depending upon the ligands employed and as indicated in the CellSearch technology publications, the next step employed is to subject the pleural fluid-magnetic particle mixture to a magnetic field to produce a cell fraction enriched in magnetic particle-bound cancer cells, if present in the pleural fluid.

In certain embodiments, depending upon the cellular contents and debris in the pleural fluid sample, the method also employs the step of purifying or separating from the enriched fraction, the non-cancer cells, non-nucleated cells, cell debris and unbound material prior to analysis. Such a purifying step can include, or be followed by, the optional step of adding to the enriched fraction a secondary ligand specific for an antigen present on non-cancer cells, e.g., WBC, present in pleural fluid. In another embodiment, the method can be modified by adding to the enriched fraction a secondary ligand specific for an antigen present on proteins normally present in the pleural fluid. Still other reagents capable of distinguishing non-cancer cells from other cells or cellular debris in the enriched fraction are added to the sample or enriched fraction to permit separation of unwanted components from the enriched fraction.

In one embodiment, such secondary ligands can include a monoclonal antibody or fragment thereof that binds specifically to, e.g., white blood cells present in the pleural fluid, among which is anti-CD45. Other antibodies unique to leukocytes or differentially expressed on leukocytes and non-leukocytes may be selected from those known in the art for this purpose. In another embodiment, the secondary ligand includes an antibody to a different determinant on the cancer cell, e.g., the labeling reagent, used in the CellSearch system that binds to, e.g., an intracellular cytokeratin of the epithelial cancer cell type or another ligand known to be present on the same cancer cell type (see, e.g., the determinants of Table 1 and the documents cited herein). Table 1 is a list of exemplary antibodies for use as the ligand/labeling agents useful in the methods described herein, and their cell determinants.

TABLE 1

| | |
|---|---|
| | MONOCLONAL |
| ANTIGEN | ANTIBODIES |

Adhesion Molecules

| | |
|---|---|
| Fibronectin receptor (α5β1 integrin) | Pierce 36114, BTC 21/22 |
| | Calbiochem 341649 |
| Integrin α3β1 | M-Kiol 2 |
| Vitronectin receptor (αγβ3 integrin) | TP36.1, BTC 41/42 |
| Integrin α2 | Calbiochem 407277 |
| Integrin α3 | Calbiochem 407278 |
| Integrin α4 | Calbiochem 407279 |
| Integrin α5 | Calbiochem 407280 |
| Integrin αV | Calbiochem 407281 |
| Integrin β2 | Calbiochem 407283 |
| Integrin β4 | Calbiochem 407284 |
| GpIIβIIIα | 8221 |
| ICAM-I (CD54) | C57-60, CL203.4, RR 1/1 |
| VCAM-1 | Genzyme 2137-01 |
| ELAM-1 | Genzyme 2138-01 |
| E-selectin | BBA 8 |
| P-selectin/GMP-140 | BTC 71/72 |
| LFA-3 (CD58) | TS 2/9 |
| CD44 | BM 1441 272, 25.32 |
| CD44-variants | 11.24, 11.31, 11.10 |
| N-CAM(CD56) | MOC-1 |
| H-CAM | BCA9 |
| L-CAM | BM 1441 892 |
| N-CAM | TURA-27 |
| MACAM-1 | NRI-M9 |
| E-cadherin | BTC 111, HECD-1, 6F9 |
| P-cadherin | NCC-CAD-299 |
| Tenascin | BM 1452 193, Calbiochem |
| | 580664 |
| Thrombospondin receptor (CD36) | BM 1441 264 |
| VLA-2 | VLA-2 |
| Lamlin receptor: HNK-1 epitope | HNK-1 |

Carbohydrate antigens

| | |
|---|---|
| T-antigen | HH8, HT-8 |
| Tn-antigen | TKH6, BaGs2 |
| Sialyl Tn | TKH-2 |
| Gastrointestinal cancer-associated antigen | CA19-9 |
| (Mw 200 kD) | |
| Carcinoma associated antigen | C-50 |
| Le^{y} | MLuC1, BR96, BR64 |
| Dimeric Le¹ epitope | NCC-ST-421 |
| H-type 2 | B1 |
| CA15-3 epitope | CA15-3 |
| CEA | I-9, I-14, I-27, II-10, |
| | I-46, Calbiochem 250729 |
| Galb1-4GlcNac (nL4, 6, 8) | 1B2 |
| H-II | BE2 |
| A type 3 | HH8 |
| Lacto-N-fucopentanose III (CD15) | PM-81 |

Glycolipids

| | |
|---|---|
| GD3 | ME 36.1, R24 |
| GD2 | ME36.1, 3F8, 14.18 |
| Gb3 | 38-13 |
| GM3 | M2590 |
| GM2 | MKI-8, MKI-16 |
| FucGM1 | 1D7, F12 |

Growth factor receptors

| | |
|---|---|
| EGF receptor | 425.3, 2.E9, 225 |
| c-erbB-2 (HER2) | BM 1378 988, 800 E6 |
| PDGFα receptor | Genzyme 1264-00 |
| PDGFβ receptor | Sigma P 7679 |
| Transferrin receptor | OKT 9, D65.30 |
| NGF receptor | BM 1198 637 |
| IL-2 receptor (CD25) | BM 1295 802, BM 1361 937 |
| c-kit | BM 428 616, 14 A3, ID9.3D6 |
| TNF-receptor | Genzyme 1995-01, PAL-M1 |
| NGF receptor | |

Melanoma antigens

| | |
|---|---|
| High molecular weight antigen (HMW | 9.2.27, NrML5, 225.28, |
| 250.000) | 763.74, TP41.2, IND1 |
| Mw 105 melanoma-associated | ME20 |
| glycoprotein | |
| 100 kDa antigen (melanoma/carcinoma) | 376.96 |
| gp 113 | MUC 18 |
| p95-100 | PAL-M2 |
| Sp75 | 15.75 |
| gr 100-107 | NKI-bereb |
| MAA | K9.2 |
| Mw 125 kD (gp125) | Mab 436 |

Sarcoma antigens

| | |
|---|---|
| TP-1 and TP-3 epitope | TP-1, TP-3 |
| Mw 200 kD | 29-13, 29.2 |
| Mw 160 kD | 35-16, 30-40 |

Carcinoma markers

| | |
|---|---|
| MOC-31 epitope (cluster 2 epithelial | MOC-31, NrLu10 |
| antigen) | |
| MUC-1 antigens (such as DF3-epitope | MUC-1, DF3, BCP-7 to -10 |
| (gp290 kD)) | |
| MUC-2 and MUC-3 | PMH1 |
| LUBCRU-G7 epitope (gp 230 kD) | LUBCRU-G7 |
| Prostate specific antigen | BM 1276 972 |
| Prostate cancer antigen | E4-SF |
| Prostate high molecular antigen Mw > | PD41 |
| 400 kD | |
| Polymorphic epithelial mucins | BM-2, BM-7, 12-H-12 |
| Prostate specific membrane antigen (Cyt- | 7E11-C5 |
| 356) | |
| Human milk fat globulin | Immunotech HMFG-1, 27.1 |
| 42 kD breast carcinoma epitope | B/9189 |
| Mw > 10⁶ mucin | TAG-72, CC-49, CC-83 |
| Ovarian carcinoma OC125 epitope (Mw | OC125 |
| 750 kD) | |
| Pancreatic HMW glycoprotein | DU-PAN-2 |
| Colon antigen Co17-1A (Mw 37000) | 17-1A G9-epitope (colon |
| | carcinoma) |
| G9 Human colonic sulfomucin | 91.9H |
| Mw 300 kD pancreas antigen | MUSE11 |
| GA 733.2 | GA733, KS1.4 |
| TAG 72 | B72.3, CC49, CC83 |
| Undefined | Oat1, SM1 |
| Pancreatic cancer-associated | MUSE 11 |
| Pancarcinoma | CC49 |
| Prostate adenocarcinoma-antigen | PD 41 |
| Mw 150-130 kD adenocarcinoma of lung | AF-10 |
| gp160 lung cancer antigen (Cancer Res. | anti gp160 |
| 48, 2768, 1988) | |
| Mw 92 kD bladder carcinoma antigen | 3G2-C6 |
| Mw 600 kD bladder carcinoma antigen | C3 |
| Bladder carcinoma antigen (Cancer Res. | AN43, BB369 |
| 49, 6720, 1989) | |
| CAR-3 epitope Mw > 400 kD | AR-3 |
| MAM-6 epitope (C15.3) | 115D8 |
| High molecular ovarian cancer antigen | OVX1, OVX2 |
| Mucin epitope Ia3 | Ia3 |
| Hepatocellular carcinoma antigen Mw | KM-2 |
| 900 kD | |
| Hepernal epitope (gp43) | |
| Hepatocellular carcinoma antigen. ag | Hepema-1 |
| O-linked mucin containing N- | 3E1.2 |
| glycolylneuraminic acid | |
| Mw 48 kD colorectal carcinoma antigen | D612 |
| Mw 71 kD breast carcinoma antigen | BCA 227 |
| 16.88 epitope (colorectal carcinoma | 16.88 |
| antigen) | |
| CAK1 (ovarian cancers) | K1 |
| Colon specific antigen p | Mu-1, Mu-2 |
| Lung carcinoma antigen Mw 350-420 kD | DF-L1, DF-L2 |
| gp54 bladder carcinoma antigen | T16 |
| gp85 bladder carcinoma antigen | T43 |
| gp25 bladder carcinoma antigen | T138 |

Neuroblastoma antigens

| | |
|---|---|
| Neuroblastoma-associated, such as | UJ13A |
| UJ13A epitope | |

Glioma antigens

| | |
|---|---|
| Mel-14 epitope | Mel-14 |

Head and neck cancer antigens

| | |
|---|---|
| Mw 18-22 kD antigen | E48 |

HLA-antigens

| | |
|---|---|
| HLA Class 1 | TP25.99 |
| HLA-A | VF19LL67 |
| HLA-B | H2-149.1 |
| HLA-A2 | KS1 |
| HLA-ABC | W6.32 |
| HLA-DR, DQ, DP | Q 5/13, B 8.11.2 |
| β2 -microglobulin | NAMB-1 |

Apoptosis receptor

| | |
|---|---|
| Apo-1 epitope | Apo 1 |

Various

| | |
|---|---|
| Plasminogen activator antigens & | Rabbit polyclonal |
| receptors | |
| p-glycoprotein | C219, MRK16, JSB-1, |
| | 265/F4 |
| cathepsin D | CIS-Diagnostici, Italy |
| biliary epithelial antigen | HEA 125 |
| neuroglandular antigen (CD63) | ME491, NKI-C3, LS62 |
| CD9 | TAPA-1, R2, SM23 |
| pan-human cell antigen | pan-H |
| Claudin 4 | |
| CD146 | |
| Vimentin | |
| B-Catenin | |
| Cytokeratin 5 | |
| Cytokeratin 6 | |
| Cytokeratin 7 | |
| Mesothelin | |
| Survivin | |
| Telomerase | |

In still a further embodiment of the purifying or separating step, a cell-specific dye, such as DAPI, is employed to stain cell nuclei to distinguish viable cells from cell debris. These may be selected as taught in the CellSearch technology publications and patents. Thus these additional ligands and reagents aide in distinguishing cancer cells from WBCs, other non-cancer cells and cell debris in the enriched fraction and permit the removal of these pleural fluid components from the enriched fraction. These additional ligands and reagents can also aide in distinguishing one cancer cell type from another.

As described above and in the examples, the common ligands and reagents used in the CellSearch system for the analysis of blood can be used for the pleural fluid analysis of certain epithelial cell cancers that are present in, or metastasize to, the pleural space in a pleural effusion. In the practice of this method, the pleural fluid sample is contacted with anti-EpCAM antibody-coated colloidal metal particles which bind and identify the cells as epithelial cancer cells. As disclosed above, the anti-EpCAM antibody coated particles can be replaced or supplemented with anti-L1CAM antibody coated particles and/or anti-Claudin 4-antibody coated particles. The sample-particle mixture is also contacted with the dye, DAPI, which stains intact cells and not cell debris. The sample-particle mixture is also contacted with anti-cytokeratin (CK) antibodies, which also identifies most epithelial cells, i.e., including the epithelial cancer cells. The sample-particle mixture is further contacted with an anti-leukocyte antibody, such as anti-CD45, to bind to the WBCs in the pleural fluid and permit their exclusion as leukocytes. Each of the antibody ligands may be coupled to a suitable label, as described in the CellSearch technology and patents describing same, which are incorporated herein by reference.

The enriched fraction, optionally purified of other components of the pleural fluid, is then analyzed to detect the number of cells characterized by the appropriate phenotype identified by the ligand/label/dye binding. As exemplified by the Example 3 herein, an epithelial cancer cell such as NSCLC, breast cancer, ovarian cancer is exemplified by a count or enumeration of cancer cells characterized by the phenotype: EpCAM⁺, DAPI⁺ CK⁺ CD45⁻. This analysis is performed using conventional immunoflow cytometry or other cell analysis and enumeration techniques identified in the CellSearch patents and publications. In one embodiment, the analysis or cancer cell enumeration of the method involves detecting or identifying or measuring a first ligand-bound cancer cell number in excess of a baseline threshold. The baseline threshold for the identified selected ligands, labeling reagents, and dyes may be routinely determined by performing the method using the same reagents in normal pleural fluids.

In one embodiment, the baseline threshold when the method employs anti-EpCAM as the first ligand is a count ranging from between about 100 to over 1000 EpCAM+, DAPI+ CK+ CD45− cells per ml of normal pleural fluid. In one embodiment, the baseline threshold when the method employs anti-EpCAM as the first ligand is a count ranging from between about 100 to over 1000 EpCAM+, DAPI+ CK+ CD45− cells per 3.5 ml of normal pleural fluid. In another embodiment, the average baseline threshold when the method employs anti-EpCAM as the first ligand is a count of about 200 EpCAM+, DAPI+ CK+ CD45− cells per ml of normal pleural fluid. In another embodiment, the average baseline threshold when the method employs anti-EpCAM as the first ligand is a count of about 300 EpCAM+, DAPI+ CK+ CD45− cells per ml of normal pleural fluid. In another embodiment, the average baseline threshold when the method employs anti-EpCAM as the first ligand is a count of about 400 EpCAM+, DAPI+ CK+ CD45− cells per ml of normal pleural fluid. In another embodiment, the average baseline threshold when the method employs anti-EpCAM as the first ligand is a count of about 500 EpCAM+, DAPI+ CK+ CD45− cells per ml of normal pleural fluid. In another embodiment, the average baseline threshold when the method employs anti-EpCAM as the first ligand is a count of about 600 EpCAM+, DAPI+ CK+ CD45− cells per ml of normal pleural fluid. In another embodiment, the average baseline threshold when the method employs anti-EpCAM as the first ligand is a count of about 700 EpCAM+, DAPI+ CK+ CD45− cells per ml of normal pleural fluid. In another embodiment, the average baseline threshold when the method employs anti-EpCAM as the first ligand is a count of about 800 EpCAM+, DAPI+ CK+ CD45− cells per ml of normal pleural fluid. In another embodiment, the average baseline threshold when the method employs anti-EpCAM as the first ligand is a count of about 900 EpCAM+, DAPI+ CK+ CD45− cells per ml of normal pleural fluid. In another embodiment, the average baseline threshold when the method employs anti-EPCAM as the first ligand is a count of more than 1000 EpCAM+, DAPI+ CK+ CD45− cells per ml of normal pleural fluid. In another embodiment, the average baseline threshold when the method employs anti-EPCAM as the first ligand is a count of more than 1100 EpCAM+, DAPI+ CK+ CD45− cells per ml of normal pleural fluid. In another embodiment, the average baseline threshold when the method employs anti-EPCAM as the first ligand is a count of more than 1100 EpCAM+, DAPI+ CK+ CD45− cells per 3.5 ml of normal pleural fluid.

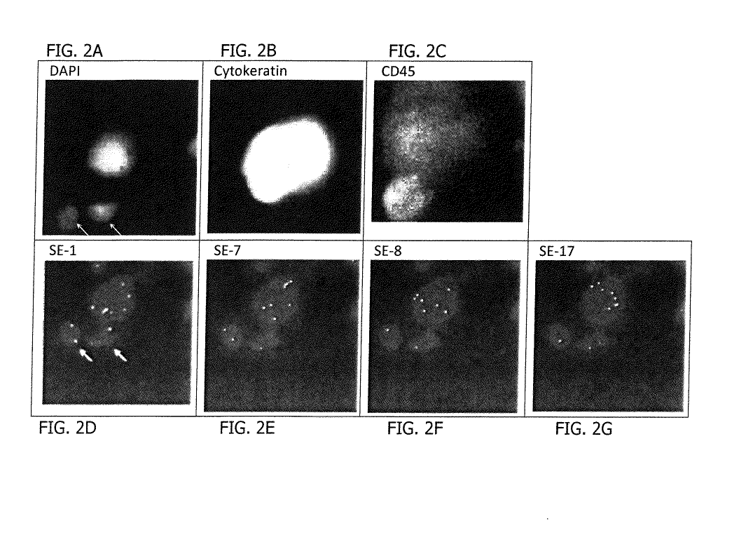

This high EpCAM⁺ baseline threshold makes it surprising that this method works in pleural fluid, because the lining of the pleurae is epithelial, and would be expected to have high EpCAM⁺ cell numbers. However, as demonstrated in Example 3, FIG. 2 and in Tables 2A, 2B and 3 below, the method described herein using anti-EpCAM as the first/capture ligand produces results that contribute to a more accurate differential diagnosis of malignancy in pleural effusions containing epithelial cell cancers. The method described in Example 3 provided a more sensitive detection of cancer in pleural effusions than did cytological evaluation of Example 2 and reached the opposite result in two breast cancer patients when using a baseline cutoff of 100 CTC/ml of pleural fluid. These results demonstrate the value of the present method of diagnosing or assisting in the differential diagnosis of cancer in pleural effusions.

In a similar manner, where the ligands, labeling agents and dyes are different from those exemplified in Example 3, and where such reagents permit one to distinguish cancer cell type, e.g., lung cancer, the baseline threshold for each different ligand is similarly calculated and the diagnostic values are determined.

In yet a further embodiment, all of these steps of the diagnostic method for analyzing the number of cancer cells in pleural fluid may be repeated at different times during the subject's disease, or prior to or during treatment of the subject's cancer, to provide information permitting an accurate diagnosis or prognosis. This diagnostic information provided by the method is likely to be most useful when combined with other conventional diagnostic methods appropriate for pleural fluids. For example, the method may be coupled with other diagnostic steps, e.g., contacting the pleural fluid sample from a subject with a diagnostic reagent that measures a level of protein or albumen in said sample.

In still another embodiment, additional diagnostic method steps may include conducting chromosomal or fluorescent in situ hybridization (FISH) analysis on isolated cancer cells from pleural fluid to determine if these have chromosomal abnormalities consistent with a neoplastic origin.

In another embodiment, nucleic acids may be analyzed from cancer cells enriched from pleural fluids, as in Example 5. Presence or absence of particular molecular markers or cytological markers may provide diagnostic or prognostic information about the patient or may help characterize the population of enriched tumor cells. Additionally, nucleic acid analysis of pleural effusion tumor cells may be used as a discovery tool to identify markers useful in managing patients or stratifying candidates for clinical trials.

As used herein, the term “cytological marker” means a marker that can be detected as part of an intact cell or fragment at the time of analysis. Methods to detect such cytological markers include but are not limited to immune-fluorescence microscopy, immune-cytochemistry, flow cytometry, FISH, and immune-fluorescent quantification. Molecular markers are substances derived from enriched PETCs that are detected following sample homogenization or purification of subcellular components of cells or cell fragments. Such markers include but are not limited to DNA, RNA, micro RNA, protein, carbohydrates, and lipids. Such markers are detected by methods which include but are not limited to PCR, RT-PCR, microarray, ELISA, western blot, and southern blot.

These methods are particularly useful in circumstances in which the subject is being treated for cancer and wherein the method enables a determination of the efficacy of the treatment or prognosis. These methods are useful in circumstances in which a subject's pleural effusion was determined to be of unknown etiology following inconclusive cytological or immunocytochemical examination of a sample of pleural fluid from the subject. These methods are also likely to be useful in the diagnoses of lung cancer, lymphoma, mesothelioma, metastatic breast cancer, metastatic ovarian cancer, and metastatic prostate cancer, among others.

### D. EMBODIMENTS OF THE METHODS

Various embodiments of the methods thus include the following.

A method for diagnosing or differentially diagnosing a cancer characterized by the presence of cancer cells in the pleural fluid or serous fluid of a mammalian subject comprises: a) contacting a sample of pleural fluid or serous fluid of the subject with colloidal magnetic particles coupled to a first ligand which binds to a determinant on cancer cells, but does not bind above a baseline threshold to other cellular and non-cellular components in pleural fluid or serous fluid; and b) subjecting the pleural fluid-magnetic particle mixture or serous fluid-magnetic particle mixture to a magnetic field to produce a cell fraction enriched in ligand-coupled, magnetic particle-bound cells, if present in the pleural fluid or serous fluid. In one embodiment, the method further includes the steps of c) analyzing the enriched fraction for the number of ligand-coupled, magnetic particle-bound cells in the pleural fluid or serous fluid; and d) providing a differential diagnosis of a cancer by identifying a number of ligand-coupled, magnetic particle-bound cells greater than the baseline threshold in the pleural fluid or serous fluid.

In still another embodiment, the method involves performing one or more additional step comprising: characterizing the enriched cell fraction using cytological or molecular markers; culturing the enriched cell fraction; and characterizing the enriched cell fraction using either cytological or molecular markers. In still another embodiment, the method includes the additional steps of analyzing the cultured enriched fraction; and performing pharmacokinetic studies on the cultured enriched fraction.

In one embodiment, the pleural fluid is a pleural effusion.

The method can also include such steps as performing cytologic or immunocytologic examination of the pleural or serous fluid for the presence of abnormal cells before or after analyzing the pleural or serous fluid by the contacting, subjecting and analysis steps. The method can include diluting the pleural or serous fluid prior to said contacting step. The pleural or serous fluid can be diluted with diluent in a ratio of 1:10. The contacting step of the method occurs within 24 hours of withdrawing the pleural fluid sample or serous fluid sample from the subject.

Additional embodiments of the methods include one or more additional steps comprising: adding to the pleural or serous fluid-magnetic particle mixture a secondary ligand specific for a second antigen expressed in cancer cells or non-cancer cells or a reagent that is capable of distinguishing non-cancer cells from other cells or cellular debris in the enriched fraction; adding to the pleural or serous fluid-magnetic particle mixture a labeling reagent that binds a second cell determinant of the cancer cell; adding to the pleural or serous fluid-magnetic particle mixture a cell-specific dye that distinguishes viable cells from cell debris; and/or purifying from the enriched fraction non-cancer cells, non-nucleated cells, cell debris and unbound material prior to analysis. In one embodiment, the secondary antibody can bind a cell determinant present on the cell surface or expressed within the cell in question.

In certain aspects of the method, the first ligand is a monoclonal antibody or fragment thereof specific for at least one cancer cell determinant. In other aspect, the secondary ligand binds specifically to white blood cells in the pleural fluid or binds to a specific tumor marker on cancer cells to enhance specificity or identify cancer cells present in the pleural or serous fluid. In other aspects, at least one of the first ligand, secondary ligand or labeling reagent binds specifically to a lung cancer cell or to a breast cancer cell. In some embodiments of the method, the first ligand binds specifically to an epithelial cell adhesion molecule (EpCAM); or the first ligand binds specifically to L1 cell adhesion molecule (L1CAM) or the first ligand binds specifically to Claudin 4. In another embodiment wherein the first ligand binds specifically to EpCAM or L1CAM and the secondary ligand binds specifically to Claudin 4.

In certain embodiments, the baseline threshold is about 1100 EpCAM⁺ cells/3.5 ml of pleural fluid. In other embodiments, the EPCAM+ cells are stained for one or more additional specific tumor markers, such as Cytokeratin, Claudin 4, survivin and/or telomerase.

In other embodiments, the method comprises one or more steps comprising: performing a clinical evaluation of the mammalian subject for clinical symptoms selected from the group consisting of chest pain, coughing, difficulty breathing, fatigue, and inflammation; contacting the pleural fluid or serous fluid sample from a subject with a diagnostic reagent that measures a level of protein or albumin in said sample; and repeating said previous steps for analyzing the number of cancer cells in pleural fluid or serous fluid at different times during the subject's disease, or prior to or during treatment of the subject's cancer. In certain embodiments, the subject is being treated for cancer and the method enables a determination of the efficacy of the treatment or prognosis. In certain embodiments, the pleural effusion was determined to be of unknown etiology following inconclusive cytologic or immunocytochemical examination of a sample of pleural fluid from the subject prior to the contacting step.

In other embodiments of the method, additional steps are selected from the following: filtering the sample of serous fluid or pleural fluid through a filter comprising pores of essentially a uniform size prior to the contacting step; analyzing the enriched cell fraction employing prognostic and predictive markers; analyzing the enriched cell fraction employing mutational analysis of genes; and/or performing fluorescence in situ hybridization of the enriched cell fraction. The prognostic and predictive markers are selected from the group consisting of EGFR, ER, Ki67, PR, Her2/nu, BCL2, M30, Cox-2, PTEN, IGF-1R, AKT, PARP, CMET, P53, P27, CEA, AR, PSMA, and PSA. Alternatively, the genes are selected from the group consisting of EGFR, BRAF, ARAF, K-ras, and P53.

Another method of diagnosing or differentially diagnosing a cancer characterized by the presence of cancer cells in the pleural fluid or serous fluid of a mammalian subject includes the steps of a) filtering a sample of pleural fluid or serous fluid of the subject with a filter comprising pores of a known essentially uniform pore size to enrich pleural effusion tumor cells (PETCs); b) analyzing the number of filtered PETCs; and c) providing a differential diagnosis of a cancer by identifying a number of filtered PETCs greater than the baseline threshold in the pleural fluid or serous fluid. In other embodiments, the method includes one or more steps comprising: analyzing the enriched cell fraction employing prognostic and predictive markers; analyzing the enriched cell fraction employing mutational analysis of genes; and performing fluorescence in situ hybridization of the enriched cell fraction. The prognostic and predictive markers are selected from the group consisting of EGFR, ER, Ki67, PR, Her2/nu, BCL2, M30, Cox-2, PTEN, IGF-1R, AKT, PARP, CMET, P53, P27, CEA, AR, PSMA, and PSA. Alternatively, the genes are selected from the group consisting of EGFR, BRAF, ARAF, K-ras, and P53.

### E. EXAMPLES

The examples that follow do not limit the scope of the embodiments described herein. One skilled in the art will appreciate that modifications can be made in the following examples which are intended to be encompassed by the spirit and scope of the invention.

### Example 1

### Pleural Fluid Sample

### I. Collection

Pleural Fluid is usually collected in a sterile 50 cc syringe or sterile urine collection container for cytological examination and can be further treated as in steps II and III, if it is intended to be stored or shipped before being examined by cytological methods as in Example 2.

For the CellSearch assay of Example 3, unprocessed pleural fluid is withdrawn, as described above. The unprocessed fluid, optionally simply diluted 1:10 with sterile buffer, is transferred to a CellSave® tube.

The collected fluid in either instance is labeled with sample number and diagnosis, if any. Optimally, fluid is forwarded to the laboratory for processing within 30 minutes of being drawn. All procedures should be done sterilely in the biosafety hood. The pleural fluid samples are subject to the assay of Example 3 within 24 hours.

### II. Optional Centrifugation Steps and Supernatant Harvesting for Cytological Examination or Storage Prior to the Method of Example 3

A. All specimens to be collected are transferred into 50 cc centrifuge tubes.

B. This material is centrifuged at ˜1,500 rpm for 10 minutes

C. About 50 ml of the supernatant is transferred to a new 50 ml centrifuge tube to harvest supernatant.

D. Centrifuge the supernatant from Step IIC at 2200 rpm for 10 minutes (This high speed spin will pellet cell debris).

E. Label 10 1.8 ml cryotubes with the sample ID, Date prepared, and sample type (“PF Supernatant”).

F. Transfer the supernatant from the high speed spin to a new 50 ml tube.

G. Aliquot 1 ml supernatant into the cryotubes.

H. Store all harvested supernatant aliquots at −80° C.

III. Resuspension of Cell Pellet from Step IIB.

A. Carefully remove and discard the remainder of the supernatant, using the vacuum aspirator, being careful not to disturb the cell pellet.

B. Re-suspend all cell pellets in a 50 ml tube with 0.5% FBS containing sterile PBS, such that the final volume is ˜20 ml.

C. Centrifuge at 1500 rpm for 5 minutes, and remove the supernatant.

D. Dilute the stock of 10× lysing buffer (BD Pharm Lyse™, Cat#555899, 10× concentrated solution) by diluting 1:10 in sterile water. Add approximately 10 times the volume of the cell pellet of 1× lysing buffer to the cell pellet, and put the tube on the orbital shaker for 15 minutes with gentle agitation. Extend the lysing step if needed

E. Centrifuge again at 1500 rpm for 5 minutes, discard the supernatant, and re-suspend with 5 ml of sterile PBS (with 0.5% FBS).

F. Remove a small aliquot and count the number of cells.

G. Transfer 2×10⁶ cells into 4 ml of PBS (with 0.5% FBS)— see step 4.

H. Transfer 1 cc of the re-suspended cell pellet to a new sterile centrifuge tube and add 6 cc of sterile PBS.

This cell suspension is evaluated in conventional cytological microscopic examination as described in Example 2.

### Example 2

### Cytologic Examination

### I. Preparing the Samples

A. Centrifuge the remaining cell suspension from Example 1, step III, subparagraph I, at 1500 rpm for another 5 minutes.

B. Remove the supernatant and prepare to add freezing media.

C. Re-suspend the pellet in 4 cc of freezing media.

D. Labels should contain sample number, sample collection date, and an indication that the sample is pleural fluid.

E. Store in a Nalgene “Mr. Frosty” freezing container containing isopropanol overnight at −80° C. Transfer cryotubes to −140° C. at that time.

### II. Cytological Analysis Using a CytoSpin® Apparatus

A. Transfer 2×10⁶ cells into 4 ml of PBS (with 0.5% FBS).

B. Pre-label the slides.

C. Prepare the slides mounted with the paper pad and the cuvette in the metal holder.

D. Load up to 200 μl of this suspension in each cuvette.

E. Spin at 750 rpm for 10 min.

F. Carefully detach the cuvette and the paper without damaging the fresh cytospin.

G. Mark the area around the cytocentrifuged cells with dry point or permanent marker.

H. Proceed with either immediate fixation or drying. Store unfixed cytospins for max 2 days at room temperature. Alternatively, store unfixed cytospins at −20C freezer for weeks.

The resulting fixed cells are examined microscopically for the presence of abnormal cells indicative of tumor cells or immature blood cells. This type of analysis produced the preliminary diagnoses recorded in column 3 of Table 1 below.

### Example 3

### Assay Using the CellSearch® System

The CellSearch® Circulating Tumor Cell system (Veridex LLC) is used to identify and enumerate the number of circulating tumor cells in a pleural fluid. The assay procedure is as described in Mayo Medical Laboratories Communique, Volume 36, No. 1 (Jan/February 2011), incorporated by reference herein, and summarized briefly below.

A. 7.5 ml of unprocessed and undiluted pleural fluid is placed in a CellSave tube (Veridex LLC).

B. The PBS is removed, and the cellular component is mixed with new buffer to a desired dilution, and then ferrofluid is added. The ferrofluid consists of nanoparticles with a magnetic core surrounded by a polymeric layer coated with antibodies targeting the Epithelial Cell Adhesion Molecule (EpCAM). The ferrofluid/antibody complex attaches specifically to epithelial cells in the cellular component.

C. The tube containing the ferrofluid/antibody complex is incubated in a magnetic cuvette, which attracts the ferrofluid-bound epithelial cells to the side of the tube. The remaining fluid and unbound cells are aspirated and the magnets are removed. The bound cells are then resuspended in buffer, resulting in an epithelial-enriched fluid.

D. Three (3) cellular staining agents are added to the epithelial-enriched fluid to help distinguish epithelial cells from contaminating leukocytes and non-specific debris:
i. 4′-6-diamidino-2-phenylindole (DAPI) is used to stain the nuclei of the cells to help identify viable cells;
ii. Phycoerythrin (PE)-labeled cytokeratin (CK) antibodies (CK 8, 18, and 19) recognize epithelial cells; and
iii. Allophycocyanin (APC)-labeled CD45 antibodies bind contaminating leukocytes.

E. The fluid is placed in the MagNest® (Veridex) cell presentation device, which attracts the magnetically-labeled epithelial cells to the surface of the cartridge. The cartridge is placed in the CellTracks® Analyzer (Veridex) and analyzed using fluorescence-based microscopy. The surface of the cartridge is scanned to acquire images of cells stained with the three agents described above.

F. Any epithelial cancer cells (e.g., EpCAM⁺ and CK⁺) present in the pleural fluid sample are identified in the images based on the phenotype: EpCAM⁺, DAN⁺ CK⁺ CD45⁻. Positive DAPI staining distinguishes viable cells from cell debris; positive CK staining distinguishes epithelial cells from other cells. The absence of CD45 staining indicates that the cells are non-leukocytes. The EpCAM+ cells indicate malignant epithelial cancer cells. All other staining combinations do not correlate to cancer cells.

Tables 2A and 2B below summarize the preliminary results of this method when masked pleural fluid samples from two cohorts of patients with pleural effusions received clinical diagnoses (which can be a combination of one or more of cytology, clinical symptomology, and/or biopsy results). These patients' samples were then subjected to the cell analysis of the method described herein using the same ligands and labeling reagents as currently used in measuring CTC in blood using the CellSearch system (e.g., identifying the cell phenotype as EpCAM+, DAPI+ CK+CD45−). The second and third columns of Tables 2A and 2B indicate the diagnosis of the masked sample. The fifth column of Tables 2A and 2B indicates the cancer cell number of the method. The fourth column shows the result from the cytology laboratory. The cohorts are identified by subject ID #, in Table 2A as CTCPF numbers and in Table 2B as UPCC numbers.

TABLE 2A

| | Diagnosis | | Cytologi- | EpCAM+, |
|---|---|---|---|---|
| Subject | based on | | cal Exam | DAPI+ |
| ID | Clinical | Sample | Results | CK+ CD45− |
| CTCPF# | Evaluation | Type | Only | CTC #/ml |
| 014 | Congestive | Benign | Negative | 2 |
| | heart failure | | | |
| 010 | Drug Reaction | Benign | Negative | 39 |
| 013 | Hepatic | Benign | Negative | 2 |
| | Hydrothorax | | | |
| 009 | Lung Transplant | Benign | Undetermined | 3 |
| 008 | Post-Radiation | Benign | Negative | 61 |
| | Effusion | | | |
| 016 | Mesothelioma | Malignant | Undetermined | 4 |
| 018 | Mesothelioma | Malignant | Undetermined | 2 |
| 012 | Multiple | Malignant | Positive | 28 |
| | Myeloma | | | |
| 011 | Breast CA | Malignant | Positive | 437 |
| 019 | Breast CA | Malignant | Negative | 1,000 |
| 021 | Breast CA | Malignant | Positive | 5,049 |
| 005 | NSCLC | Malignant | Negative | 8 |
| 015 | NSCLC | Malignant | Positive | 667 |
| 017 | NSCLC | Malignant | Undetermined | 1,333 |
| 006 | Ovarian CA | Malignant | Positive | 952 |
| 022 | Renal Cell CA | Malignant | Negative | 0 |

TABLE 2B

| | Diagnosis | | Cytologi- | EpCAM+, |
|---|---|---|---|---|
| Subject | based on | | cal Exam | DAPI+ |
| ID | Clinical | Sample | Results | CK+ CD45− |
| UPCC # | Evaluation | Type | Only | CTC #/ml |
| 23510-001 | NSCLC - ADC | Malignant | Positive | 42,857 |
| 23510-002 | Pending | | Undetermined | 2 |
| 23510-003 | Renal Cell CA | Malignant | Negative | 19 |
| 23510-004 | Radiation | Benign | Negative | 80 |
| | induced PE | | | |
| 23510-005 | Pending | | Negative | 37 |
| 23510-006 | NSCLC - NOS | Malignant | Positive | 163,610 |
| 23510-007 | Renal Cell CA | Malignant | Negative | 1 |
| 23510-008 | Breast CA | Malignant | Positive | 84,848 |
| 23510-009 | Pending | | Undetermined | 9 |
| 23510-010 | Parapneumonic | Benign | Negative | 57 |
| | Effusion | | | |
| 23510-011 | Breast CA | Malignant | Negative | 213 |
| 23510-012 | Hepatic | Benign | Negative | 7 |
| | Hydrothorax | | | |
| 23510-013 | NSCLC - ADC | Malignant | Positive | 148 |
| 23510-014 | Thymic CA- | Malignant | Positive | 4042 |
| | Squamous | | | |
| 23510-015 | NSCLC - NOS | Malignant | Positive | 28,821 |
| 23510-016 | Pancreatic | Malignant | Positive | 5949 |
| | AdenoCA | | | |
| 23510-017 | Renal Cell CA | Malignant | Positive | 6457 |
| 23510-018 | CHF | Benign | Negative | 56 |
| 23510-019 | Lymphoma | Malignant | Positive | 0 |
| 23510-020 | CHF | Benign | Negative | 34 |
| 23510-021 | Gastric ADC | Malignant | Negative | 431 |
| 23510-022 | Radiation | Benign | Negative | 0 |
| | induced PE | | | |
| 23510-023 | Small Cell | Malignant | Positive | 11749 |
| | Lung Cancer | | | |
| 23510-024 | NSCLC- | Malignant | Positive | 307738 |
| | Squamous | | | |
| 23510-025 | Pending | | Negative | 133 |
| 23510-026 | CHF | Benign | Negative | 12 |
| 23510-027 | NSCLC-NOS | Malignant | Positive | 68211 |
| 23510-028 | Pending | | Suspicious | 83 |
| 23510-029 | Breast CA | Malignant | Positive | 231 |
| 23510-030 | Parapneumonic | Benign | Negative | 30 |
| | Effusion | | | |
| 23510-031 | Malignant | Malignant | Positive | 755 |
| | Mesothelioma | | | |
| 23510-032 | Hepatic | Benign | Negative | 195 |
| | Hydrothorax | | | |
| 23510-033 | Breast CA | Malignant | Positive | 87097 |
| 23510-034 | Rectal CA | Malignant | Positive | 291 |
| 23510-035 | Thoracic Duct | Malignant | Negative | 2 |
| | Rupture | | | |
| 23510-036 | Pending | | Negative | 1 |
| 23510-037 | Gastric ADC | Malignant | Positive | 1797 |
| 23510-038 | Transudative | Benign | Negative | 0 |
| | Effusion- NOS | | | |
| 23510-039 | Pending | | Negative | 1 |
| 23510-040 | Pending | | Negative | 1047 |
| 23510-041 | Pending | | Negative | 16 |
| 23510-042 | CHF | Benign | Negative | 7 |
| 23510-043 | Malignant | Malignant | Positive | 526 |
| | Melanoma | | | |
| 23510-044 | NSCLC-NOS | Malignant | Negative | 37 |
| 23510-045 | Lymphoma- | Malignant | Negative | 1 |
| | Mantle Cell | | | |
| 23510-046 | Pending | | Suspicious | 22 |
| 23510-047 | Pending | | Negative | 27 |
| 23510-048 | Renal Cell CA | Malignant | Undetermined | 12827 |
| 23510-049 | NSCLC-ADC | Malignant | Negative | 56 |
| 23510-050 | CHF | Benign | Negative | 3 |
| 23510-051 | CHF | Benign | Negative | 45 |
| 23510-052 | ESRD | Benign | Negative | 0 |
| 23510-053 | Esophageal ADC | Malignant | Positive | 65714 |
| 23510-054 | Thyroid CA | Malignant | Negative | 13 |
| 23510-055 | Small Cell | Malignant | Positive | 280 |
| | Lung Cancer | | | |
| 23510-056 | Pancreatic | Malignant | Negative | 154 |
| | AdenoCA | | | |
| 23510-057 | Pending | | Positive | 1543 |
| 23510-058 | Pending | | Negative | 7 |
| 23510-059 | NSCLC-ADC | Malignant | Positive | 502 |
| 23510-060 | PE - NOS | Benign | Negative | 4 |
| 23510-061 | NSCLC- | Malignant | Negative | 120 |
| | Squamous | | | |
| 23510-062 | Pending | | Negative | 16 |
| 23510-063 | Pending | | Negative | 96 |
| 23510-064 | Yellow Nail | Benign | Negative | 13 |
| | Syndrome | | | |
| 23510-065 | Pending | | Negative | 17 |
| 23510-066 | Pending | | Negative | 1 |
| 23510-067 | Pending | | Negative | 19 |
| 23510-068 | Pending | | Negative | 8 |
| 23510-069 | Pending | | Suspicious | 111 |

Tables 3A and 3B below summarize the updated preliminary results of this method when masked pleural fluid samples from two cohorts of patients with pleural effusions received clinical diagnoses (which can be a combination of one or more of cytology, clinical symptomology, and/or biopsy results). These patients' samples were then subjected to the cell analysis of the method described herein using the same ligands and labeling reagents as currently used in measuring CTC in blood using the CellSearch system (e.g., identifying the cell phenotype as EpCAM+, DAPI+ CK+CD45−). The second and third columns of Tables 3A and 3B indicate the diagnosis of the masked sample. The fifth column of Tables 3A and 3B indicates the cancer cell number of the method. The fourth column shows the result from the cytology laboratory. The cohorts are identified by subject ID #, in Table 3A as CTCPF numbers and in Table 3B as UPCC numbers.

TABLE 3A

| | DIAGNOSIS | | CYTOLOGI- | EPCAM+, |
|---|---|---|---|---|
| SUBJECT | BASED ON | | CAL EXAM | DAPI+ |
| ID | CLINICAL | SAMPLE | RESULTS | CK+ CD45− |
| CTCPF# | EVALUATION | TYPE | ONLY | CTC #/ML |
| 014 | Congestive | Benign | Negative | 2 |
| | heart failure | | | |
| 010 | Drug Reaction | Benign | Negative | 39 |
| 013 | Hepatic | Benign | Negative | 2 |
| | Hydrothorax | | | |
| 009 | Lung Transplant | Benign | Undetermined | 3 |
| 008 | Post-Radiation | Benign | Negative | 61 |
| | Effusion | | | |
| 016 | Mesothelioma | Malignant | Undetermined | 4 |
| 018 | Mesothelioma | Malignant | Undetermined | 2 |
| 012 | Multiple | Malignant | Positive | 28 |
| | Myeloma | | | |
| 011 | Breast CA | Malignant | Positive | 437 |
| 019 | Breast CA | Malignant | Negative | 1,000 |
| 021 | Breast CA | Malignant | Positive | 5,049 |
| 005 | NSCLC | Malignant | Negative | 8 |
| 015 | NSCLC | Malignant | Positive | 667 |
| 017 | NSCLC | Malignant | Undetermined | 1,333 |
| 006 | Ovarian CA | Malignant | Positive | 952 |
| 022 | Renal Cell CA | Malignant | Negative | 0 |

TABLE 3B

| | | SAMPLE | CTC/3.5 | |
|---|---|---|---|---|
| SUBJECT ID | DIAGNOSIS | TYPE | ML FLUID | CYTOLOGY |
| UPCC-23510-001 | NSCLC - ADC | Malignant - | 150000 | Positive |
| | | Epithelial | | |
| UPCC-23510-002 | Pending | | 7 | Undetermined |
| UPCC-23510-003 | Renal Cell Carcinoma | Malignant - | 66.5 | Negative |
| | | Epithelial | | |
| UPCC-23510-004 | Radiation induced | Benign | 278.5 | Negative |
| | pleural effusion | | | |
| UPCC-23510-005 | Pending | | 130 | Negative |
| UPCC-23510-006 | NSCLC - NOS | Malignant - | 572635 | Positive |
| | | Epithelial | | |
| UPCC-23510-007 | Renal Cell Carcinoma | Malignant - | 2 | Negative |
| | | Epithelial | | |
| UPCC-23510-008 | Breast CA | Malignant - | 296969 | Positive |
| | | Epithelial | | |
| UPCC-23510-009 | NSCLC - ADC | Malignant - | 32 | Negative |
| | | Epithelial | | |
| UPCC-23510-010 | Parapneumonic | Benign | 198 | Negative |
| | Effusion | | | |
| UPCC-23510-011 | Breast CA | Malignant - | 745 | Negative |
| | | Epithelial | | |
| UPCC-23510-012 | Hepatic Hydrothorax | Benign | 23 | Negative |
| UPCC-23510-013 | NSCLC - ADC | Malignant - | 519 | Positive |
| | | Epithelial | | |
| UPCC-23510-014 | Thymic Carcinoma - | Malignant - | 14147 | Positive |
| | Squamous Cell | Epithelial | | |
| UPCC-23510-015 | NSCLC - NOS | Malignant - | 100874 | Positive |
| | | Epithelial | | |
| UPCC-23510-016 | Pancreatic | Malignant - | 20821 | Positive |
| | Adenocarcinoma | Epithelial | | |
| UPCC-23510-017 | Renal Cell Carcinoma | Malignant - | 22600 | Positive |
| | | Epithelial | | |
| UPCC-23510-018 | CHF | Benign | 197 | Negative |
| UPCC-23510-019 | Lymphoma | Malignant - | 4 | Positive |
| | | Non- | | |
| | | Epithelial | | |
| UPCC-23510-020 | CHF | Benign | 120 | Negative |
| UPCC-23510-021 | Gastric ADC | Malignant - | 1509 | Negative |
| | | Epithelial | | |
| UPCC-23510-022 | Radiation induced | Benign | 0 | Negative |
| | pleural effusion | | | |
| UPCC-23510-023 | Small Cell Lung | Malignant - | 41120 | Positive |
| | Cancer | Epithelial | | |
| UPCC-23510-024 | NSCLC - Squamous | Malignant - | 1077082 | Positive |
| | | Epithelial | | |
| UPCC-23510-025 | Radiation induced | Benign | 467 | Negative |
| | pleural effusion | | | |
| UPCC-23510-026 | CHF | Benign | 41 | Negative |
| UPCC-23510-027 | NSCLC - NOS | Malignant - | 238740 | Positive |
| | | Epithelial | | |
| UPCC-23510-028 | Pending | | 289 | Suspicious |
| UPCC-23510-029 | Breast CA | Malignant - | 808 | Positive |
| | | Epithelial | | |
| UPCC-23510-030 | Parapneumonic | Benign | 104 | Negative |
| | Effusion | | | |
| UPCC-23510-031 | Malignant | Malignant - | 2642 | Positive |
| | Mesothelioma | Non- | | |
| | | Epithelial | | |
| UPCC-23510-032 | Hepatic Hydrothorax | Benign | 681 | Negative |
| UPCC-23510-033 | Breast CA | Malignant - | 304840 | Positive |
| | | Epithelial | | |
| UPCC-23510-034 | Rectal CA | Malignant - | 1018 | Positive |
| | | Epithelial | | |
| UPCC-23510-035 | Thoracic Duct Rupture | Benign | 8 | Negative |
| UPCC-23510-036 | Cardiac Surgery | Benign | 3 | Negative |
| UPCC-23510-037 | Gastric ADC | Malignant - | 6291 | Positive |
| | | Epithelial | | |
| UPCC-23510-038 | Transudative | Benign | 0 | Negative |
| | Effusion - NOS | | | |
| UPCC-23510-039 | BAPE | Benign | 2 | Negative |
| UPCC-23510-040 | Pending | | 3663 | Negative |
| UPCC-23510-041 | Pending | | 56 | Negative |
| UPCC-23510-042 | CHF | Benign | 24.5 | Negative |
| UPCC-23510-043 | Malignant Melanoma | Malignant - | 1840 | Positive |
| | | Epithelial | | |
| UPCC-23510-044 | NSCLC - NOS | Malignant - | 128 | Negative |
| | | Epithelial | | |
| UPCC-23510-045 | Lymphoma - Mantle | Malignant - | 4 | Negative |
| | Cell | Non- | | |
| | | Epithelial | | |
| UPCC-23510-046 | Pending | | 76 | Pending |
| UPCC-23510-047 | Radiation induced | Benign | 94 | Negative |
| | pleural effusion | | | |
| UPCC-23510-048 | Renal Cell Carcinoma | Malignant - | 44894 | Negative |
| | | Epithelial | | |
| UPCC-23510-049 | NSCLC - ADC | Malignant - | 197 | Negative |
| | | Epithelial | | |
| UPCC-23510-050 | CHF | Benign | 10 | Negative |
| UPCC-23510-051 | CHF | Benign | 157 | Negative |
| UPCC-23510-052 | ESRD | Benign | 0 | Negative |
| UPCC-23510-053 | Esophageal ADC | Malignant - | 230000 | Positive |
| | | Epithelial | | |
| UPCC-23510-054 | Thyroid CA | Malignant - | 47 | Negative |
| | | Epithelial | | |
| UPCC-23510-055 | Small Cell Lung | Malignant - | 979 | Positive |
| | Cancer | Epithelial | | |
| UPCC-23510-056 | Pancreatic | Malignant - | 540 | Negative |
| | Adenocarcinoma | Epithelial | | |
| UPCC-23510-057 | Pending | | 5400 | Pending |
| UPCC-23510-058 | Malignant | Malignant - | 25 | Negative |
| | Mesothelioma | Non- | | |
| | | Epithelial | | |
| UPCC-23510-059 | NSCLC - ADC | Malignant - | 1757 | Positive |
| | | Epithelial | | |
| UPCC-23510-060 | Pleural Effusion - | Benign | 13 | Negative |
| | NOS | | | |
| UPCC-23510-061 | NSCLC - Squamous | Malignant - | 421 | Negative |
| | | Epithelial | | |
| UPCC-23510-062 | Pending | | 55 | Negative |
| UPCC-23510-063 | Pending | | 334 | Negative |
| UPCC-23510-064 | Yellow Nail | Benign | 47 | Negative |
| | Syndrome | | | |
| UPCC-23510-065 | Post-operative | | 61 | Negative |
| | Effusion | | | |
| UPCC-23510-066 | Pending | | 3.5 | Pending |
| UPCC-23510-067 | Renal Cell Carcinoma | Malignant - | 67 | Negative |
| | | Epithelial | | |
| UPCC-23510-068 | ESRD | Benign | 41 | Negative |
| UPCC-23510-069 | Ampullary Carcinoma | Malignant - | 416 | Negative |
| | | Epithelial | | |
| UPCC-23510-070 | Pericardial Sarcoma | Malignant - | 36 | Positive |
| | | Non- | | |
| | | Epithelial | | |
| UPCC-23510-071 | ESRD | Benign | 200 | Negative |
| UPCC-23510-072 | Malignant Melanoma | Malignant - | 1246 | Positive |
| | | Epithelial | | |
| UPCC-23510-073 | Radiation induced | Benign | 3 | Negative |
| | pleural effusion | | | |
| UPCC-23510-074 | Cholangiocarcinoma | Malignant - | 6 | Negative |
| | | Epithelial | | |
| UPCC-23510-075 | Small Cell Lung | Malignant - | 1551 | Positive |
| | Cancer | Epithelial | | |
| UPCC-23510-076 | NSCLC - ADC | Malignant - | 245200 | Positive |
| | | Epithelial | | |
| UPCC-23510-077 | Breast CA | Malignant - | 6221 | Positive |
| | | Epithelial | | |
| UPCC-23510-078 | Ovarian CA | Malignant - | 7251 | Positive |
| | | Epithelial | | |
| UPCC-23510-079 | Pending | | 14 | Negative |
| UPCC-23510-080 | Pending | | 5 | Negative |
| UPCC-23510-081 | Rectal CA | Malignant - | 513 | Positive |
| | | Epithelial | | |
| UPCC-23510-082 | NSCLC - ADC | Malignant - | 2848 | Positive |
| | | Epithelial | | |
| UPCC-23510-083 | Amyloidosis | Benign | 0 | Negative |
| UPCC-23510-084 | Pending | | 18 | Negative |
| UPCC-23510-085 | NSCLC - ADC | Malignant - | 470 | Positive |
| | | Epithelial | | |
| UPCC-23510-086 | Breast CA | Malignant - | 505 | Positive |
| | | Epithelial | | |
| UPCC-23510-087 | Pending | | 1485 | Negative |
| UPCC-23510-088 | Malignant Melanoma | Malignant - | 174 | Positive |
| | | Epithelial | | |
| UPCC-23510-089 | Small Cell Lung | Malignant - | 200 | Positive |
| | Cancer | Epithelial | | |
| UPCC-23510-090 | Malignant | Malignant - | 2057 | Negative |
| | Mesothelioma | Non- | | |
| | | Epithelial | | |
| UPCC-23510-091 | Transudative | Benign | 2 | Negative |
| | Effusion - NOS | | | |
| UPCC-23510-092 | NSCLC - ADC | Malignant - | 2338 | Negative |
| | | Epithelial | | |
| UPCC-23510-093 | Pending | | 47 | Negative |
| UPCC-23510-094 | Granulomatous | Benign | 0 | Negative |
| | Pleuritis | | | |
| UPCC-23510-095 | Breast CA | Malignant - | 379 | Negative |
| | | Epithelial | | |
| UPCC-23510-096 | Ovarian CA | Malignant - | 1400000 | Positive |
| | | Epithelial | | |
| UPCC-23510-097 | Pending | | 85 | Negative |
| UPCC-23510-098 | Pending | | 2219 | Negative |
| UPCC-23510-099 | Lymphoma - Mantle | Malignant - | 226 | Positive |
| | Cell | Non- | | |
| | | Epithelial | | |
| UPCC-23510-100 | Pleural Effusion - | Benign | 181 | Negative |
| | NOS | | | |
| UPCC-23510-101 | NSCLC - NOS | Malignant - | 24 | Positive |
| | | Epithelial | | |
| UPCC-23510-102 | NSCLC - NOS | Malignant - | 5279 | Positive |
| | | Epithelial | | |
| UPCC-23510-103 | Pending | | 27 | Positive |
| UPCC-23510-104 | Breast CA | Malignant - | 295550 | Positive |
| | | Epithelial | | |
| UPCC-23510-105 | Pending | | 1 | Negative |
| UPCC-23510-106 | Thyroid Carcinoma - | Malignant - | 9738 | Positive |
| | Papillary | Epithelial | | |
| UPCC-23510-107 | Lymphoma | Malignant - | 77 | Positive |
| | | Non- | | |
| | | Epithelial | | |
| UPCC-23510-108 | Uterine CA | Malignant - | 1850 | Positive |
| | | Epithelial | | |
| UPCC-23510-109 | ESRD | Benign | 215 | Negative |
| UPCC-23510-110 | Lymphoma | Malignant - | 135 | Negative |
| | | Non- | | |
| | | Epithelial | | |
| UPCC-23510-111 | NSCLC - NOS | Malignant - | 0 | Positive |
| | | Epithelial | | |
| UPCC-23510-112 | Pancreatic | Malignant - | 168800 | Positive |
| | Adenocarcinoma | Epithelial | | |
| UPCC-23510-113 | Renal Cell Carcinoma | Malignant - | 130 | Negative |
| | | Epithelial | | |
| UPCC-23510-114 | NSCLC - Squamous | Malignant - | 7762 | Negative |
| | | Epithelial | | |
| UPCC-23510-115 | Malignant | Malignant - | 311 | Positive |
| | Mesothelioma | Non- | | |
| | | Epithelial | | |
| UPCC-23510-116 | NSCLC - ADC | Malignant - | 1762 | Positive |
| | | Epithelial | | |
| UPCC-23510-117 | Pending | | 64 | Negative |
| UPCC-23510-118 | Lymphoma | Malignant - | 1830.5 | Positive |
| | | Non- | | |
| | | Epithelial | | |
| UPCC-23510-119 | Pending | | 191 | Negative |
| UPCC-23510-120 | Pending | | 61 | Negative |
| UPCC-23510-121 | NSCLC - NOS | Malignant - | 20312 | Negative |
| | | Epithelial | | |
| UPCC-23510-122 | Transudative | Benign | 5 | Negative |
| | Effusion - NOS | | | |
| UPCC-23510-123 | Malignant | Malignant - | 1209 | Negative |
| | Mesothelioma | Non- | | |
| | | Epithelial | | |
| UPCC-23510-124 | Pending | | 80 | Negative |
| UPCC-23510-125 | Pending | | 2981 | N/A |
| UPCC-23510-126 | Pending | | 15 | Negative |
| UPCC-23510-127 | Pending | | 33 | N/A |
| UPCC-23510-128 | Adenocarcinoma-NOS | Malignant - | 807 | Negative |
| | | Epithelial | | |
| UPCC-23510-129 | Pending | | 11 | Negative |
| UPCC-23510-130 | NSCLC - ADC | Malignant - | 1105000 | Positive |
| | | Epithelial | | |
| UPCC-23510-131 | Renal Cell Carcinoma | Malignant - | 707 | Positive |
| | | Epithelial | | |
| UPCC-23510-132 | Pending | | 2 | Negative |
| UPCC-23510-133 | CHF | Benign | 28 | Negative |
| UPCC-23510-134 | Pending | | 25 | Negative |
| UPCC-23510-135 | Pending | | 865 | Negative |
| UPCC-23510-136 | Pending | | 70 | Negative |
| UPCC-23510-137 | Pending | | 2 | Negative |
| UPCC-23510-138 | CHF | Benign | 11 | Negative |
| UPCC-23510-139 | Pending | | 101 | Negative |
| UPCC-23510-140 | Bladder Cancer | Malignant - | 227 | Negative |
| | | Epithelial | | |
| UPCC-23510-141 | Pending | | 22 | Negative |
| UPCC-23510-142 | Pending | | 934 | Negative |
| UPCC-23510-143 | Pending | | 16 | Negative |
| UPCC-23510-144 | NSCLC - ADC | Malignant - | 795432 | Positive |
| | | Epithelial | | |
| UPCC-23510-145 | Peritoneal Dialysis | Benign | 101 | Negative |
| UPCC-23510-146 | NSCLC - Squamous | Malignant - | 623 | Positive |
| | | Epithelial | | |
| UPCC-23510-147 | Breast CA | Malignant - | 792837 | Positive |
| | | Epithelial | | |
| UPCC-23510-148 | Chylothorax | Benign | 1364 | Negative |
| UPCC-23510-149 | Esophageal ADC | Malignant - | 263098 | Positive |
| | | Epithelial | | |
| UPCC-23510-150 | Breast CA | Malignant - | 7833 | Positive |
| | | Epithelial | | |
| UPCC-23510-151 | Pending | | 236 | Negative |

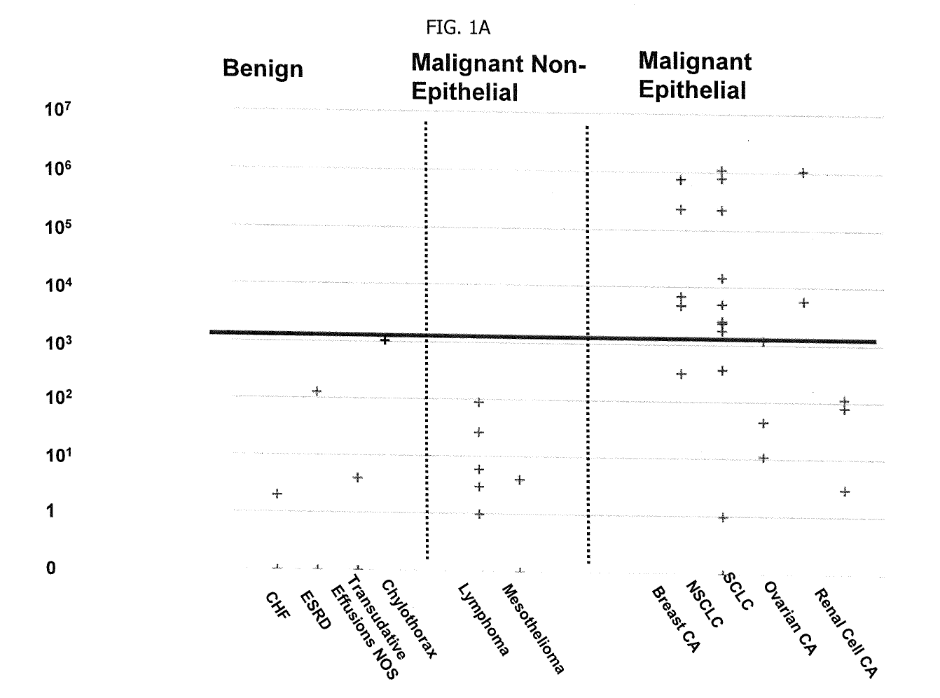

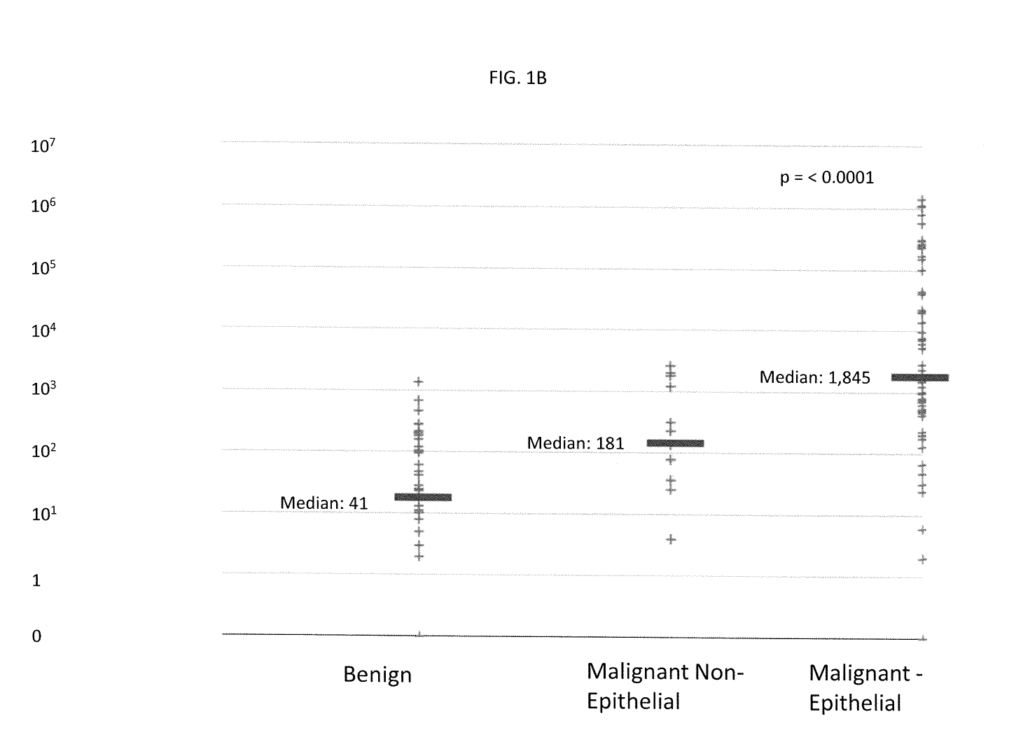

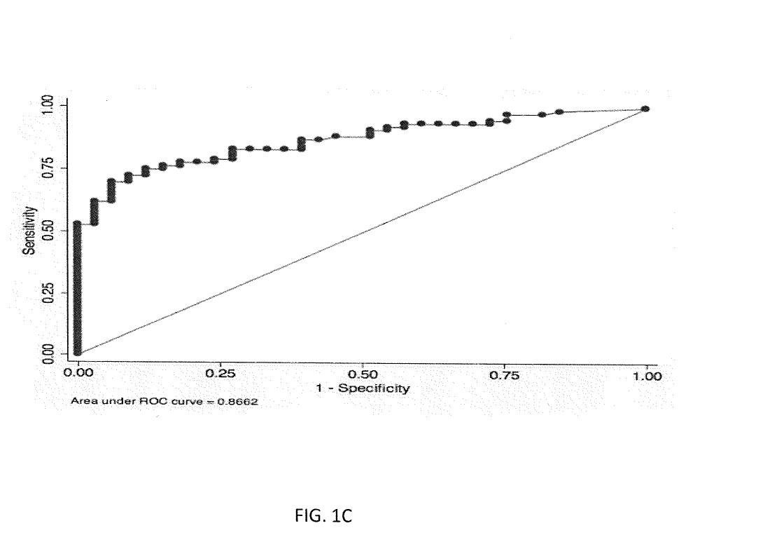

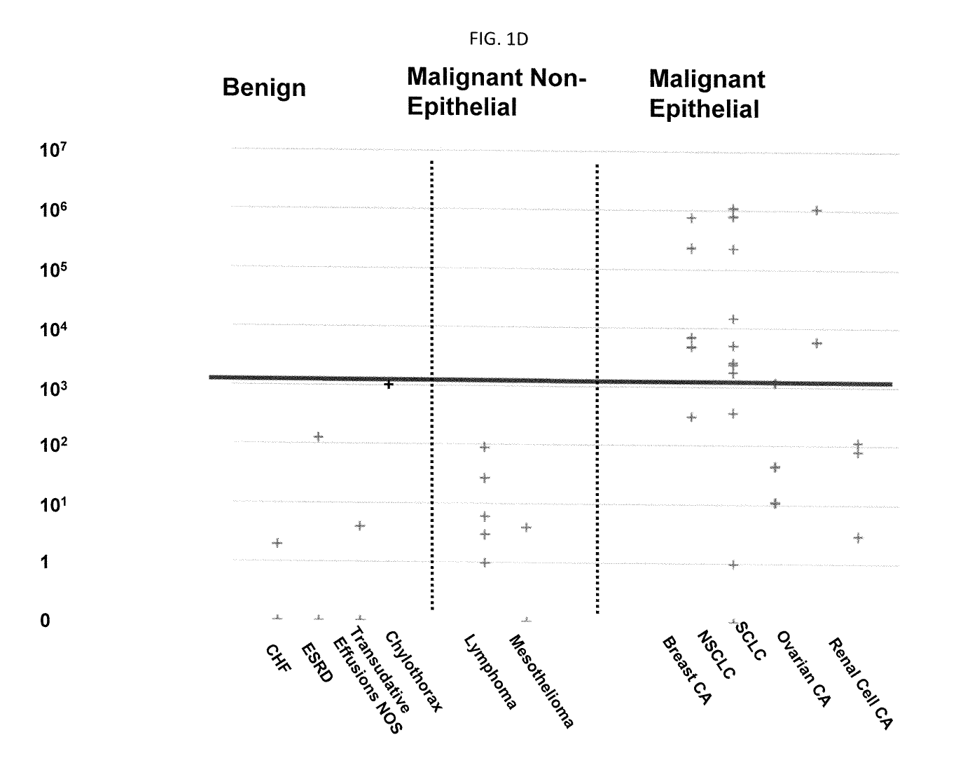

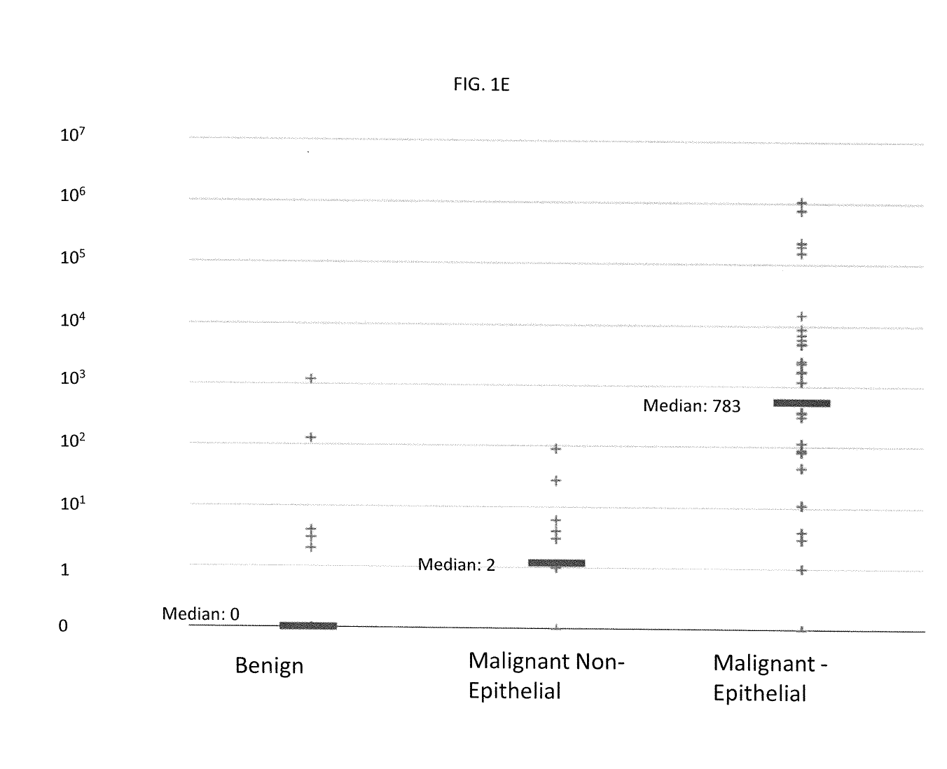

For samples of non-malignancy, e.g., CHF, drug reaction, hepatic hydrothorax, lung transplant and post-radiation, most of these cell number results (Col. 4, Table 3B) were well under the 1100 cell/3.5 ml pleural fluid baseline threshold, the average number was 141. For non-epithelial cell cancer patient samples (mesothelioma, and possibly renal cell and thymic cell), and the hematological cancer, multiple myeloma (believed to be all cancer cells not bearing the EpCAM determinant), the counts for each patient were often under the baseline of 1100 and averaged 181. The average count per 3.5 ml for the epithelial malignancies was 1,845. FIG. 1B shows this data.

The diagnostic accuracy of the test depends on the thresholds chosen. This threshold is able to be readily determined by one of skill in the art as indicated by additional data. Higher thresholds result in higher specificity with less sensitivity. From the data collected so far (Tables 3A and 3B), this can be seen in the receiver operating curve shown in FIG. 1C and shown in Table 4 below.

TABLE 4

| | | | Correct |
|---|---|---|---|
| Cutoff | Sensitivity | Specificity | Classification |
| 2 | 98.7% | 15.5% | 73.4% |
| 311 | 72.4% | 90.9% | 78% |
| 1509 | 57.8% | 100% | 72.1% |

Not all of the cancer patients have very high levels. Many patients had cell numbers over 100 cell/ml PF. It is presently suspected that the tumors in the patients with lower counts may have lost the CK determinant, or chemotherapy reduced the number of tumor cells in the pleural effusion, or an advanced stage of cancer in which the tumor cells had undergone the epithelial mesenchymal transformation, and lost the EpCAM⁺ determinant. Additionally, for those samples showing high average cell counts (see Table 4), it is presently theorized that unlike measuring CTC in blood samples, the higher cell counts do not necessarily indicate a worse stage of cancer.

As noted in Tables 2A and 2B, for patients CTCPF-019 and UPPCC-23510-011, the cytological evaluation demonstrated that the pleural fluids samples from these patients were negative for malignancy. The method described in Example 3 provided a more sensitive detection than did cytological evaluation of Example 2 and reached the opposite result in these two patients when using a baseline cutoff of 100 CTC/3.5 ml of pleural fluid. These results demonstrate the value of the present method of diagnosing or assisting in the differential diagnosis of cancer in pleural effusions.

Table 5 below summarizes the average number of tumor cells (CTC) per 3.5 ml of pleural fluid sorted by diagnosis for the cohort UPCC of Table 3B. The graph of FIG. 1A illustrates this data in a scatter plot representation of the number of CTC per 3.5 ml of each subject according to etiology and the current figures support the original data.

TABLE 5

| | | |
|---|---|---|
| | AVERAGE | NUMBER OF |
| CONDITION | CTC/3.5 ML | SUBJECTS |

Benign

| | | |
|---|---|---|
| Amyloidosis | 0 | 1 |
| CHF | 74 | 8 |
| Chylothorax | 1364 | 1 |
| Empyema | | |
| ESRD | 114 | 2 |
| Granulomatous Pleuritis | 0 | 1 |
| Hepatic Hydrothorax | 352 | 2 |
| Parapneumonic Effusion | 151 | 2 |
| Peritoneal Dialysis | 101 | 1 |
| Pleural Effusion - NOS | 97 | 2 |
| Radiation Pleuritis | 169 | 3 |
| Thoracic Duct Rupture | 8 | 1 |
| Transudative Effusion - NOS | 2 | 3 |
| Yellow Nail Syndrome | 42 | 1 |

Malignant Epithelial

| | | |
|---|---|---|
| Ampullary CA | 416 | 1 |
| Breast CA | 170669 | 10 |
| Esophageal CA | 246549 | 2 |
| Malignant Melanoma | 1087 | 3 |
| NSCLC - ADC | 192130 | 11 |
| NSCLC - NOS | 133999 | 8 |
| NSCLC - SCC | 271472 | 2 |
| Ovarian CA | 703626 | 2 |
| Pancreatic ADC | 63387 | 3 |
| Rectal CA | 766 | 2 |
| Renal Cell CA | 9781 | 6 |
| Small Cell Lung CA | 10963 | 4 |
| Thymic CA | 14147 | 1 |
| Thyroid CA | 9738 | 1 |
| Uterine CA | 1850 | 1 |

Malignant - Non-Epithelial

| | | |
|---|---|---|
| Lymphoma | 512 | 2 |
| Mantle Cell Lymphoma | 115 | 2 |
| Malignant Mesothelioma | 1249 | 4 |
| Pericardial Sarcoma | 36 | 1 |

As noted above in the data in the tables above, the method described in Example 3 provided a more accurate and sensitive detection and diagnosed malignancy for patients for whom the cytological evaluation of Example 2 was indefinite or incorrect.

Preliminary data using a second staining marker (Claudin 4) to lower the background staining levels were also collected. The tight protein-associated Claudin4 (CL4) has been shown to be present in malignant cells of epithelial origin, but not in mesothelial cells. In PF cytology, it can be difficult to differentiate reactive mesothelial cells from those of malignant origin. Cells that were EPCAM+/cytokeratin+/CD45−/DAPI+ were then stained for Claudin 4 in approximately 80 patients. Background levels of staining were markedly reduced. The median value for benign disease was 0 cells/3.5 ml, for malignant non-epithelial tumors was 2 cells/3.5 ml, and for malignant epithelial tumors was 783 cells/3.5 ml. FIGS. 1D and 1E show the distribution of cell counts versus diagnoses.

### Example 4

### Characterization of Tumor Cells Using CellSearch® System

In this example, tumor cells are enumerated and characterized with various biomarkers. The CellTracks® Analyzer II is a 4 color microscope which uses 3 colors for enumeration of tumor cells and the fourth color is available to characterize tumor cells with biomarker of interest. The biomarkers tested in this example are Ki67, EGFR, Muc1, Ki67, CD44, CEA, CD146, beta catenin, mesothelin, and cytokeratins 5, 6 and 7. The CellSearch® Circulating Tumor Cell system (Veridex LLC) is used to identify and enumerate the number of circulating tumor cells in a pleural fluid. The sample preparation for tumor cell characterization is same as described in Example 3 except that biomarker of interest conjugated to a fluorescent dye is used as marker reagent on the CellTracks® AutoPrep system.

Table 6 shows the expression of various markers on tumor cells from different patients, identified by patient sample numbers PF23501-44, -49, -51, -57, -61, -62, -63, -67, -75 and 02501-053. The marker expression varies for each patient and shows a characteristic profile for each patient. This example shows that tumor cells isolated from pleural fluid can be characterized for treatment prediction and personalized treatment selection.

TABLE 6
PERCENTAGE OF TUMOR CELLS POSITIVE WITH MARKERS

| | PATIENT SAMPLE NUMBERS | | | | | | | | | |
|---|---|---|---|---|---|---|---|---|---|---|
| MARKERS | −44 | −49 | −51 | −57 | −61 | −62 | −63 | −67 | −053 | −75 |
| Ki67 | 3 | 52 | 2 | 37 | 8 | 6 | 25 | 16 | 33 | 1 |
| EGFR1 | 20 | 1 | 47 | 14 | 73 | 36 | 6 | 43 | 42 | 76 |
| Muc1 | 98 | 53 | 99 | 56 | 78 | 27 | 38 | 96 | 97 | 97 |
| CD146 | 39 | 74 | 38 | 67 | 29 | 2 | 57 | 6 | 27 | 1 |
| CEA | 17 | 20 | 5 | 9 | 1 | 0 | 4 | 89 | 17 | 18 |
| Vimentin | 1 | 3 | 9 | 0 | 85 | 25 | 68 | 10 | 3 | 5 |
| CD44 | NA | NA | NA | NA | NA | NA | 55 | 2 | 18 | 27 |
| B-Catenin | NA | NA | 68 | 45 | 64 | 18 | 45 | 53 | 30 | 70 |
| Cytokeratin 5 | NA | 65 | 98 | 25 | 93 | 76 | 69 | 98 | 99 | 80 |
| Cytokeratin 6 | NA | NA | NA | 100 | NA | NA | 46 | 54 | 56 | 24 |
| Cytokeratin 7 | 99 | 25 | 99 | 100 | NA | NA | 87 | 99 | 97 | 1 |
| Mesothelin | NA | NA | NA | NA | NA | NA | NA | 35 | 81 | 7 |

### Example 5

### Isolation of Tumor Cells for Nucleic Acid Analysis

In this example, tumor cells are isolated from pleural effusion by CellTracks

AutoPrep system using CellSearch CTC Profile kit (Veridex LLC). The Profile kit enriches tumor cells by depleting most of the leukocytes and other blood cells from pleural fluid thereby minimizing background. The CellSearch CTC Profile Kit contains reagents only to isolate tumor cells and does not contain any staining reagents or permeabilize the cells as discussed in Example 3.

A. 7.5 ml of unprocessed and undiluted pleural fluid is placed in a CellSave tube (Veridex LLC) or EDTA tube.

B. The PBS is removed, and the cellular component is mixed with new buffer to a desired dilution, and then ferrofluid is added. The ferrofluid consists of nanoparticles with a magnetic core surrounded by a polymeric layer coated with antibodies targeting the Epithelial Cell Adhesion Molecule (EpCAM). The ferrofluid/antibody complex attaches specifically to epithelial cells in the cellular component. The tube containing the ferrofluid/antibody complex is incubated in a magnetic cuvette, which attracts the ferrofluid-bound epithelial cells to the side of the tube. The remaining fluid and unbound cells are aspirated and the magnets are removed. The bound cells are then resuspended in buffer, resulting in an epithelial-enriched fluid.

C. Following enrichment, nucleic acids fractions are isolated from PETCs for downstream analysis using techniques appropriate for the analyte and known to those skilled in the art. Nucleic acid fractions that may be isolated include, without limitation, total RNA, messenger RNA, micro RNA, and DNA.

D. Isolated nucleic acids are then analyzed using techniques appropriate for the analyte of interest. Methods of downstream analysis include, without limitation, PCR, RT-PCR, whole genome amplification, whole transcriptome amplification, DNA sequencing, RNA sequencing, mutation analysis, SNP detection, microarray analysis, and methylation status determination.

### Example 6

### Characterization of Pleural Effusion Tumor Cells by Fluorescent In Situ Hybridization

Fluorescent In Situ Hybridization (FISH) is a technique used to identify chromosomal mutations such as aneusomy, translocations, gene amplification and/or deletions. Characterizing the mutation status of tumors is useful in predicting response to therapeutic treatments, providing prognostic information about patient outcomes patient stratification for therapeutic clinical trials. This example describes how FISH analysis is carried out on PETC cells. The term FISH is intended to be used interchangeably with CISH (Chromogenic In Situ Hybridization).

A. Cells are enriched and scanned using CellSearch® technology as described in examples 3 and 4 and subsequently processed for FISH.

B. Cells are fixed to the glass surface of the cartridge by addition of methanol/acetic acid fixative and subsequently air dried. This method preserves the location of the majority of PETC cells within the cartridge. The samples may be stored at −20° C. for several years.

C. Labeled hybridization probes are applied to the sample. The sample and probe are co-denatured at 80° C. for 5 minutes then allowed to hybridize overnight at 42° C. In this example, satellite enumeration probes specific to centromere of chromosomes 1, 7, 8, and 17 are fluorescently labeled with Platinum Bright™ 647, Platinum Bright™ 550, Platinum Bright™ 505 and Platinum Bright™ 415, respectively. Platinum Bright™ kits are obtained from Kreatech, Amsterdam NL. Following hybridization, a stringent wash is used to remove non-specific hybridization and the sample is counterstained with DAPI.

D. Images of PETCs are acquired using a CellTracks® instrument that has been modified with special software, a 40× objective, and appropriate filter cubes to visualize the fluorochromes used on FISH probes. Archival data from the CellSearch scan is used to perform cartridge calibration and relocate PETC identified in the CellSearch scan. Composite images prepared from five z-stacks are acquired for each fluorochrome for each tumor cell.

FIGS. 2A-2G shows the sequential immunostaining and FISH results on same PETC, as described above.

### Example 7

### Larger Study

The initial data above suggests that this method has great diagnostic potential in terms of sensitivity and specificity. To determine the performance characteristics of the method, pleural fluid was collected from 150 subjects, all volunteers, which included both benign effusions and a variety of malignant pleural effusions. According to the methods outlined in the examples above, 7.5 ml of pleural fluid was extracted from each patient sample received at the laboratory (in cases in which more than 50 cc has been collected) and placed in a CellSave tube. The tubes were analyzed by the same techniques described in Example 3.

Clinical information related to the effusions including results of chemical analysis, cytology results, and other biopsies were collected. When a diagnosis was not made at the time of fluid collection, these subjects are being followed for up to 12 months or until a diagnosis is made.

Although all 150 samples have been collected, a relatively large number of patients are still being clinically followed. After all the data has been collected, the performance characteristics of the test are calculated using standard statistical approaches. In one embodiment, the method is modified by including additional steps, such as immunostaining with secondary antibody to a “pan-malignant” protein, such as Claudin 4, survivin or telomerase. Still additional method steps including conducting chromosomal or fluorescent in-situ hybridization (FISH) analysis on isolated cancer cells from pleural fluid to determine if these have chromosomal abnormalities consistent with a neoplastic origin.

All documents listed in this specification and U.S. Provisional Patent Application No. 61/512,576, filed Jul. 28, 2011, are incorporated herein by reference. While various embodiments in the specification or claims are presented using “comprising” language, under various circumstances, a related embodiment may also be described using “consisting of” or “consisting essentially of” language. It is to be noted that the term “a” or “an”, refers to one or more, for example, “a reagent,” is understood to represent one or more reagents. As such, the terms “a” (or “an”), “one or more,” and “at least one” are used interchangeably herein. While the invention has been described with reference to specific embodiments, it is appreciated that modifications can be made without departing from the spirit of the invention. Such modifications are intended to fall within the scope of the appended claims.

1. A method for diagnosing or differentially diagnosing a cancer characterized by the presence of cancer cells in the pleural fluid or serous fluid of a mammalian subject, the method comprising:
a) contacting a sample of pleural fluid or serous fluid of the subject with colloidal magnetic particles coupled to a first ligand which binds to a determinant on cancer cells, but does not bind above a baseline threshold to other cellular and non-cellular components in pleural fluid or serous fluid; and
b) subjecting the pleural fluid-magnetic particle mixture or serous fluid-magnetic particle mixture to a magnetic field to produce a cell fraction enriched in ligand-coupled, magnetic particle-bound cells, if present in the pleural fluid or serous fluid.

2. The method of claim 1, further comprising:
c) analyzing the enriched fraction for the number of ligand-coupled, magnetic particle-bound cells in the pleural fluid or serous fluid; and
d) providing a differential diagnosis of a cancer by identifying a number of ligand-coupled, magnetic particle-bound cells greater than the baseline threshold in the pleural fluid or serous fluid.

3. (canceled)

4. The method of claim 1, further comprising performing cytologic or immunocytologic examination of the pleural or serous fluid for the presence of abnormal cells before or after analyzing the pleural or serous fluid by the contacting, subjecting and analysis steps.

5. The method of claim 1, further comprising diluting the pleural or serous fluid prior to said contacting step, wherein the pleural or serous fluid is optionally diluted with diluent in a ratio of 1:10.

6-7. (canceled)

8. The method of claim 1, further comprising one or more of:
adding to the pleural or serous fluid-magnetic particle mixture a secondary ligand specific for an antigen expressed in cancer cells or non-cancer cells or a reagent that is capable of distinguishing non-cancer cells from other cells or cellular debris in the enriched fraction;
adding to the pleural or serous fluid-magnetic particle mixture a labeling reagent that binds a second cell determinant of the cancer cell;
adding to the pleural or serous fluid-magnetic particle mixture a cell-specific dye that distinguishes viable cells from cell debris; or
purifying from the enriched fraction non-cancer cells, non-nucleated cells, cell debris and unbound material prior to analysis.

9. The method of claim 1, wherein the first ligand is a monoclonal antibody or fragment thereof specific for at least one cancer cell determinant.

10. The method of claim 8, wherein the secondary ligand binds specifically to white blood cells in the pleural fluid or binds to a specific tumor marker on cancer cells to enhance specificity or identify cancer cells present in the pleural or serous fluid; or wherein at least one of the first ligand, secondary ligand or labeling reagent binds specifically to a lung cancer cell or to a breast cancer cell; or or wherein the first ligand binds specifically to EpCAM or L1CAM and the secondary ligand binds specifically to Claudin 4.

11. (canceled)

12. The method of claim 1, wherein the cancer is selected from the group consisting of a lung cancer, a lymphoma, mesothelioma, a metastatic breast cancer, a metastatic ovarian cancer, and a metastatic prostate cancer.

13. (canceled)

14. The method of claim 1, wherein the first ligand binds specifically to L1 cell adhesion molecule (L1CAM), to an epithelial cell adhesion molecule (EpCAM), or to Claudin 4.

15-16. (canceled)

17. The method of claim 14, wherein the baseline threshold is about 1100 EpCAM⁺ cells/3.5 ml of pleural fluid.

18. (canceled)

19. The method of claim 14, wherein the EpCAM+ cells are stained for one or more additional specific tumor markers and the additional specific tumor marker is one of Cytokeratin, Claudin 4, survivin or telomerase.

20. The method of claim 1, further comprising one or more of:
performing a clinical evaluation of the mammalian subject for clinical symptoms selected from the group consisting of chest pain, coughing, difficulty breathing, fatigue, and inflammation;
contacting the pleural fluid or serous fluid sample from a subject with a diagnostic reagent that measures a level of protein or albumin in said sample; and
repeating said steps (a) and (b) of claim 1 for analyzing the number of cancer cells in pleural fluid or serous fluid at different times during the subject's disease, or prior to or during treatment of the subject's cancer.

21-22. (canceled)

23. The method of claim 1, comprising one or more of:
filtering the sample of serous fluid or pleural fluid through a filter comprising pores of essentially a uniform size prior to the contacting step;
analyzing the enriched cell fraction employing prognostic and predictive markers;
analyzing the enriched cell fraction employing mutational analysis of genes; and
performing fluorescence in situ hybridization of the enriched cell fraction.

24. The method of claim 23, wherein the prognostic and predictive markers are selected from the group consisting of EGFR, ER, Ki67, PR, Her2/nu, BCL2, M30, Cox-2, PTEN, IGF-1R, AKT, PARP, CMET, P53, P27, CEA, AR, PSMA, and PSA.

25. The method of claim 23, wherein the genes are selected from the group consisting of EGFR, BRAF, ARAF, K-ras, and P53.

26. The method of claim 1, further comprising performing one or more of:
characterizing the enriched cell fraction using cytological or molecular markers;
culturing the enriched cell fraction;
characterizing the enriched cell fraction using either cytological or molecular markers;
analyzing the cultured enriched fraction; and
performing pharmacokinetic studies on the cultured enriched fraction.

27. (canceled)

28. A method for diagnosing or differentially diagnosing a cancer characterized by the presence of cancer cells in the pleural fluid or serous fluid of a mammalian subject, the method comprising:
a) filtering a sample of pleural fluid or serous fluid of the subject with a filter comprising pores of a known essentially uniform pore size to enrich pleural effusion tumor cells (PETCs);
b) analyzing the number of filtered PETCs; and
c) providing a differential diagnosis of a cancer by identifying a number of filtered PETCs greater than the baseline threshold in the pleural fluid or serous fluid.

29. The method of claim 28, comprising one or more of:
analyzing the enriched cell fraction employing prognostic and predictive markers;
analyzing the enriched cell fraction employing mutational analysis of genes; and
performing fluorescence in situ hybridization of the enriched cell fraction.

30. The method of claim 29, wherein the prognostic and predictive markers are selected from the group consisting of EGFR, ER, Ki67, PR, Her2/nu, BCL2, M30, Cox-2, PTEN, IGF-1R, AKT, PARP, CMET, P53, P27, CEA, AR, PSMA, and PSA.

31. The method of claim 29, wherein the genes are selected from the group consisting of EGFR, BRAF, ARAF, K-ras, and P53.
